(19) United States
(12) Patent Application Publication
(10) Pub. No.: US 2019/0200950 A1
(43) Pub. Date: Jul. 4, 2019

(54) System And Method For Non-Invasive Measurement Of Pressure Inside A Body Including Intravascular Blood Pressure

(71) Applicant: PI-Harvest Holding AG, Schaffhausen (CH)

(72) Inventors: Alexander Brenner, Haifa (IL); Yuri Brodsky, Haifa (IL)

(73) Assignee: PI-Harvest Holding AG, Schaffhausen (CH)

(21) Appl. No.: 16/322,921

(22) PCT Filed: Aug. 3, 2017
(86) PCT No.: PCT/EP2017/069756

§ 371 Date: Feb. 1, 2019

Related U.S. Application Data
(63) Continuation-in-part of application No. 15/227,905, filed on Aug. 3, 2016.

(30) Foreign Application Priority Data
Aug. 3, 2016 (EP) 16182619.3

(51) Int. Cl. A61B 8/04; A61B 8/12; A61B 8/08; A61B 8/00

(52) U.S. Cl. CPC A61B 8/04; A61B 8/12; A61B 8/488; A61B 8/486; A61B 8/58; A61B 8/5223; A61B 8/5246; A61B 8/463

(57) ABSTRACT

A system, device (140) and method for the non-invasive ultrasound or any other imaging based measurement system of the intravascular blood pressure is presented, wherein the blood pressure measurements are performed by means of the image time series processing estimating the volumes of the oscillating traceable regions. The new generalized M-mode being the set of M-modes corresponding to all ultrasound channels is introduced. The invention is applicable to any medium transparent for imaging waves capable to be converted into the image series calibrated to the pressure changes of the liquid.

### PRESENT FIELD

The present invention pertains to the field of implantable medical devices and systems as well as related methods. More particularly it relates to a medical imaging modality for the non-invasive determination of a pressure inside of a body from outside the body. The medical imaging modality may include a non-invasive medical imaging system, such as ultrasound; artificially implanted, or natural signal reflectors, such as ultrasound wave reflectors; a set of processes for pressure measurements, e.g. for measuring intravascular blood pressure; and to medical imaging transducers and receivers.

### BACKGROUND

Diseases including congestive heart failure (CHF), abdominal aortic aneurysm (AAA), pulmonary artery hypertension (PAH), are a major cause of premature death. There is a desire to be able to provide an advantageous monitoring of intravascular and/or intracardial blood pressure, including continuous monitoring. Based on such blood pressure measurements, diagnosis and treatment of patients can be based on a unique level, preventing substantial populations of patients from premature death.

Preferably, implantable sensors are that implanted via endovascular techniques.

There are active sensors which need a rechargeable energy source, which is undesired and related to a number of apparent disadvantages.

Invasive measurement of intra cardiac pressure by pressure sensors introduced on catheters is desired to be minimized due to complexity of the procedure and related patient risk. There are passive implantable sensors which are typically electromagnetic, providing an electromagnetic signal when irradiated from the external to the human body source of electromagnetic energy mainly in radio frequencies (RF). These sensors have electronics incorporated and have thus related disadvantages, such as size or reliability of the implanted sensor, over time. Moreover, while a part of the RF energy is absorbed by the implanted RF sensor, parts of the RF energy are absorbed by the body which may cause potential problems in an the living organisms. Energy transmitted from outside the body may be converted in these implants to power the electronics, make measurements and transmit measurement results to the outside of the body again. A detecting system external to the human body registers the electromagnetic field irradiated in its turn by the circuit of the implanted sensor is detected by the detecting system. An example of electromagnetic sensors is described in the U.S. Pat. No. 7,245,117 B1 with the title “Communicating with implanted wireless sensor”, the resonant frequency of a sensor is determined for energizing the system to burst the RF energy at predetermined frequencies and amplitudes. A similar technology is described in the U.S. Pat. No. 8,894,582 B2.

Intravascular ultrasound measurements are known, but restricted to either catheter based ultrasound transceivers introduced into the body, mainly for imaging and Doppler ultrasound measurements. Blood pressure in peripheral vessels may be measured non-invasively from the outside of the body using ultrasound. However, calibration of such ultrasound-based methods is complex and not always reliable. Moreover, such methods cannot selectively measure pressure at specific depths and places in the body, e.g. in the aorta or the heart. Other, non-invasive techniques include methods to examine dimensions of blood vessels, or methods based on examining blood flow and are based on Doppler ultrasound or other ultrasound imaging methods, as disclosed in, for instance, U.S. Pat. No. 5,411,028, 5,477,858 A, 5,544,656, 6,814,702 B2, 5,724,973 A, US 20140081144 A1, EP 1421905 A1, U.S. Pat. No. 7,128,713 B2, WO 2007087522 A2, US 20080119741 A1, U.S. Pat. No. 7,736,314 B2, US 20130197367 A1, or US 20130006112.

For example in U.S. Pat. No. 5,520,185 A with the title “Method for recognition and reduction of blood speckle in blood vessel imaging system”, a method for enhancing an intravascular ultrasound blood vessel image system is disclosed. It is explained how ultrasound echoes representing vessel walls are distinguished from ultrasound echoes from blood flow by using a classifier which employs the mean and variance of the raw data of grey scale intensities as acquired directly from an ultrasound scanner-detector.

In U.S. Pat. No. 5,800,356 with the title “Ultrasonic diagnostic imaging system with Doppler assisted tracking of tissue motion”, a method for tracing the border of tissue through temporarily acquired scan lines using velocity information corresponding to the tissue edges to trace the denoted border is disclosed.

In U.S. Pat. No. 6,258,031 B1 with the title “Ultrasound diagnostic apparatus”, the velocity of a blood flow and velocity of a blood walls at the same time are measured by the ultrasound with phase detecting.

In US 20090171205 A1 with the title “Method and system for locating blood vessels”, a method utilizing the direct ultrasound sounding for detecting the blood vessels and precisely determining of their depth and diameter is disclosed.

In U.S. Pat. No. 8,469,887 B2 with the title “Method and apparatus for flow parameter imaging”, a method using pulse-wave spectral Doppler imaging allowed to obtain an ultrasound image as a sectional image of the blood vessel, including the inner and outer walls.

Other methods and systems for blood pressure measurements in the blood vessels using Doppler ultrasound imaging are disclosed in: U.S. Pat. No. 5,749,364 A1, WO 20010000 A9, US 20070016037 A1, US 20050015009 A1, US 20140180114 A1, US 20140148702, U.S. Pat. No. 8,968,203 B2, US 20150289836.

In US 20150230774 A1 with the title “Blood pressure monitor and method”, non-invasive continuous real-time monitoring of an arterial blood pressure is disclosed using Doppler probes for systolic and diastolic blood pressure.

The above discussed non-invasive ultrasound or Doppler ultrasound methods for the examination of the blood vessels have a number of explicit deficiencies and it is desirous to overcome each of these deficiencies, alone or in combination. Deficiencies include but are not limited to the below:

1. Reproducibility and accuracy of the examination of the blood vessel is highly dependent on the correct orientation of the ultrasound beam's propagation direction (the axis of the ultrasound transducer) relatively to the vessel's longitudinal axis being interrogated. The speed of blood flow Measuring is measured by converting of the value of the shift of the Doppler frequency Δf using the Doppler equation:

V=(c×Δf)/(2f₀×cos α),

where: V is the velocity of the blood flow, c is the speed of sound in the tissue, f₀ is the initial frequency of the signal, and α is the angle between the direction of the blood flow and the axis of the ultrasound beam. The angle α strongly affects the value of the measured Doppler frequency Δf which in turn is used to calculate of the speed of the organic reflectors in the blood flow.

2. Reliability and precision of blood vessel examination including blood pressure measurement based on ultrasound can be improved. For instance, the Doppler frequency spectra display the blood flow information from a certain area at a given depth, (control volume), and do not provide information about blood flow in other parts of the vessel which are visible on the ultrasound image. Therefore, in case choosing an inadequate control volume (ex., when cos α−0) all diagnostic information will be incorrect.

3. Moreover, examination of blood vessels using ultrasound imaging methods carries the common drawbacks of the ultrasound diagnostic technology, such as echogenicity of objects under examination and resolution capabilities.

Insufficient accuracy of results from hemodynamic measurements in blood vessels using certain Doppler methods are well documented. For example, in: S. B. Coffi, D. Th. Ubbink and D. A. Legemate. Non-invasive Techniques to Detect Subcritical Iliac Artery Stenosis. Eur. J. Vascular and Endovascular Surgery, 29, 2005; Ricardo Cesar, Rocha Moreira. Comparative study of Doppler ultrasonography with arteriography in the evaluation of aortoiliac occlusive disease. Journal Vascular Brasileiro, 8, January/March 2009; or Vilhelm Schaberle. Ultrasonography in Vascular Diagnosis. A Therapy-Oriented Textbook and Atlas. Second Edition. Springer Heidelberg-Dordrecht-London-New-York, 2011.

The article Gernot Schulte-Altedorneburg, Dirk W. Droste, Szabolcs Felszegny, Monica Kellerman et al., Accuracy in vivo Carotid B-mode Ultrasound Compared with Pathological Analysis: Intima-Media Thickening, Lumen Diameter and Cross-Sectional Area. Stroke: Journal of the American Heart Association, 2001 demonstrates an insufficient accuracy of the results obtained for the examination of blood vessels using of the ultrasound B-mode imaging only.

There exist previous patents using passive sensors placed in the human body and interacting with an external ultrasound source for analysis of physiological parameters of the human organism, as for instance U.S. Pat. Nos. 5,619,997 A, 5,989,190 A, 6,083,165 A, or US 20030176789 A1. However, these devices and methods have a number of drawbacks, namely the following:

1. The disclosures in U.S. Pat. Nos. 5,619,997 A, 5,989,190 A, 6,083,165 A consist in the suggestion that the physical parameters (pressure, temperature, viscosity) defining the state of the medium (including the human body) are determined as a functional relationship P=f(v), where P is the physical parameter and v is the frequency of the ultrasound wave reflected by a passive sensor placed in the medium which is different from the frequency of the primary ultrasound beam due the energy absorption by the sensor.

2. The disclosure in patent application US 20030176789 A1 suggests that the value of a specific physical parameter, as the pressure, associated with the specific state of any medium (including the human body) is determined as the result of the frequency analysis of the acoustic signal reflected by the passive sensor implanted into the medium. The passive sensor has to be equipped with two parallel to each other reflective surfaces and the reflected signal is the result of the interference of the two acoustic signals: the first signal is reflected by the first reflective surface and second signal reflected by the second reflective surface.

The frequency analysis of the resultant signal permits allocate the frequencies of the maximal attenuation of the intensity and the value of the specific physical parameter is determined on the basis of the correlation relationships between the values of the parameters and the frequencies of the maximum attenuation of the resultant signal. The knowledge of the correlation relationships between the values of the parameters and the frequencies is not sufficient to determine the functional relationship P=F(v). The method is dependent of the frequencies of both the direct and reflected signals, It is desired to provide a more simple method and system that is independent of the frequencies of both the direct and reflected signals which are also present in the following patent: US 20070208293 A1 “Methods and devices for non-invasive pressure measurement in ventricular shunts”. This disclosure relates to a ventricular shunt including a pressure-sensitive body that changes its dimensions in response to the pressure of the cerebrospinal fluid within the shunt.

The difference of US 20070208293 A1 from current document lies in several aspects. First, the flow of cerebrospinal fluid is quasi-stationary unlike the turbulent blood flow such as inside the heart chambers which are dealt with in the current disclosure. Second, the system from US 20070208293 A1 is tracking the distance changes between the transducer and a ultrasonic beam reflecting gas-filled capsule, while in the current description the pressure is determined/estimated as the function of volumes of oscillating traceable regions in a series of the images, not necessarily produced by ultrasound.

On the other hand, we note the successful approach of the linear regression modeling of the maximal value of the Left Atrium pressure changes through the simultaneous measurements of the Left Atrium pressure with a catheter and trans-esophageal Doppler echocardiography published in the article “Noninvasive assessment of left atrial maximum dP/dt by a combination of transmitral and pulmonary venous flow”, see the Journal of the American College of Cardiology, V. 34, Issue 3, September 1999, P. 795-801, by Satoshi Nakatani, Mario J Garcia, Michael S Firstenberg, Leonardo Rodriguez, Richard A Grimm, Neil L Greenberg, Patrick M McCarthy. However, in this article it had not been realized that not only Doppler echocardiography but regular ultrasound imaging can be used to assess the atrial (both left and right) blood pressure and not only pressure changes. The present inventors realized this fact.

The approach in the present document is based on advantageous synchronized simultaneous measurements of the intra-cardiac blood pressure, such as with a micro-manometer catheter, and ultra-sound (as an example of a suitable image modality) recordings with subsequent signal and image processing for determination and/or estimation of intra body pressure.

### SUMMARY OF THE INVENTION

The present invention is defined by the enclosed patent claims. The present document may include basis for further separate or overlapping inventions. The advantages of the disclosed medical imaging modality over the prior art presented by this disclosure include, amongst others, alone or in combination:
Method to derive pressure data based on ultrasound measurements
Usage of passive, moving membranes (artificial or natural)
Synchronized, simultaneous measurements of intra-cardiac blood pressure with a micro-manometer catheter and an imaging device such as ultrasound
Process for the analysis of a sequence of images, in the case of ultrasound collected in the generalized M-mode, being the simultaneous analysis of all M-modes
Process for deriving pressure data as function of volume changes and for calibrating the medical imaging modality

The methods of some aspects of the disclosure comprise set of processes for pressure measurement that are based on processing time series of images obtained by the medical imaging modality to estimate volumes using oscillating traceable regions time series of images.

A system or modality is for instance provided for determining a pressure inside a body. The system includes a control unit. The control unit is configured to estimate at least a volume of an oscillating traceable region inside said body. The volume may be estimated from at least a series of images generated by an ultrasound or other medical imaging unit. The control unit is configured to correlate said volume with a pressure at said region for said determining of said pressure.

The system preferably includes at least one medical implant previously implanted at said body for tracing said oscillating region in said series of images. The implant optionally and preferably has at least one reflective surface and said surface is preferably an integral part of the implant or is attached to the implant. Most preferably, said implant is part of the group of impmants known to people skilled in the art as “passive implants” or “passive sensor”.

The medical implant is preferably implantable in the cardiovascular system, in regions including the heart, veins or arteries. Preferably said implant is implantable in the atria and most preferably said implant is implanted in the inter-atrial wall of the heart.

In another aspect of this disclosure the medical implant is implanted in major vessels of the cardiovascular system.

Most preferably said implant is implantable in the pulmonary arteries.

A plurality of such implants described herein may advantageously used for pressure determination, e.g. with improved precision of the pressure determination result and/or for multiple pressure values at different anatomical positions that may be interrelated to each other.

Said implant may include devices from the group including devices used for the repair or occlusion cardiovascular structures. For example, said implant includes devices known to people skilled in the art as occluders, plugs, coils. stents, or shunt devices. Said implants may include devices such as ASD, PFO, LAA, or paravalvular leak occluders; stenting devices intended to maintain the patency of vessels, openings (natural or induced), or cavities in cardiovascular structures; or plug devices to close, seal or obstruct cardiovascular structures.

In one aspect of this disclosure medical images of said implant are used to determine pressure. In another aspect of this disclosure medical images of a naturally occurring cardiovascular structures are used to determine pressure. In either aspect of this disclosure medical images are taken from oscillating traceable region in a non-invasive manner and from the exterior of the body. Preferably said images are taken from at least one atrium or one pulmonary artery.

A method of determining a pressure inside a body is provided. Said method includes estimating at least a volume of an oscillating traceable region inside said body from at least a series of images generated by an ultrasound or other medical imaging unit, and correlating said volume with a pressure at said region for said determining of said pressure.

A software comprising an algorithm for performing such pressure determination method is provided. Said software is preferably stored on a computer readable medium.

The present dislcosure provides systems, methods, devices, software and uses of implants that permit to directly measure pressure and its dynamic changes inside a body from the outside of the body without the need of an actively driven implanted device. For instance, measuring blood pressure and its dynamic changes within the cardiovascular system, including in a blood vessel or intracardially such as in a heart chamber, appendage etc. is facilitated by means of an implanted passive implant or passive sensor. A passive sensor may be implanted into the cardiovascular system, such as into an artery or the heart itself. The sensor is preferably implanted minimally invasively via a catheter based technique. Further, the passive sensor optionally has ultrasonic beam reflectors which reflect ultrasound waves generated by ultrasound transducers to be captured by ultrasound receivers. Alternatively, the intra-cardiac structures such as heart chambers can play the role of the passive sensors in particular if the intra body pressure is initially calibrated by alternative means as micro-manometer catheters. In the absence of the calibration only the pressure relative dynamic changes can be calculated/determined.

The present document provides herein the means for pressure measurement inside the body, such as inside the cardiovascular system, measured e.g. by ultrasound or any other medical imaging system. Calculation or determination of pressure is based on medical image time series. Said Calculation or determination includes the processing of measurements of the dynamics of a movably reflective surface portion of a passive artificial or natural (ultrasound) reflector, optionally implanted inside a body. Said reflector may be optionally implanted in the cardiovascular system, preferably in the hart or in blood vessels.

For said Calculation or determination, the pressure P will be defined as the best fit function of a given shape inside of a body: P=F(L₁, L₂) where L₁ is the brightness line of the first artificial or natural stationary surface in the image, which is preferably ultrasound image of said passive reflector and L₂ is the brightness line of a second moving artificial or natural surface of said passive reflector in the image measured at the same time moment (see e.g. Upper and Lower Paths in FIG. 20).

This present publication discloses amongst others a novel method to calculate and determine said pressure and said method is independent of the frequencies of both the direct and reflected signals. Thus, prior technical solutions, such as disclosed in U.S. Pat. Nos. 5,619,997 A, 5,989,190 A, 6,083,165 A, or US 20030176789.

High accuracy and stability of intra-body pressure measurements are preferably based on synchronised and simultaneous measurements using catheter-based pressure sensors and imaging devices followed by compiling a mathematical model to calculate the pressure function and calibrated to the measured, real-time absolute pressure values. Thus, when the system is calibrated, the (blood) pressure and its dynamic changes within the body, such as in a blood vessel can be calculated with high accuracy and stability anytime when (ultrasound) measurements are provided with the imaging (preferably ultrasound) device connected to the current system.

The essence of a preferred example of the current disclosure lies in the development of a direct method of ultrasonic measurement of the blood pressure in the heart or a blood vessel and an apparatus for its practical implementation.

The presented disclosure of a method is in an example for intravascular blood pressure measurement based on the presence of two different imaging (preferably ultrasound beam) reflecting surfaces, said reflecting surfaces may be composed of the same or differing materials and/or may have the same or different shapes:
a) A first surface or surface portion at a constant position, i.e. said first surface is independent of intravascular blood pressure changes, by virtue of it being fixed on, onto, to, or by tit being in relation to a cardiovascular wall, such as an vessel or heart chamber wall;
b) A second surface or surface portion configured so as to oscillate relative to the first surface and in relation to intravascular blood pressure changes.

Both first and second surfaces are placed into a cardiovascular structure subject to changes as result of pressure changes in or around said cardiovascular structure. Said surfaces are placed such that the first surface, or surface portion of it, and the second surface, or surface portion of it, are in fluid communication. Said fluid communications is provided by preferably a liquid such as blood. Said pressure changes preferably are blood pressure changes inside the cardiovascular system, such as inside of a blood vessel or the heart.

The present disclosure further provides an example of a system for subsequent calibration, measurements and calculations of pressure based on the volume of cardiovascular structures including but not limited to the left atrium (LA), right atrium (RA), left ventricle (LV), right ventricle (RA), or the pulmonary artery (PA). During the calibration the values Pᵢ at time moments tᵢ are measures by direct pressure meters, such as catheter based blood pressure sensors. The imaging (preferably ultra-sound) measurements are provided simultaneously by an imaging modality (preferably an ultrasound device). Both intra body pressure meter measurement data and images series over time is synchronously recorded into the system (1100) and optimally regressed to a function F of a given shape in the way that Pᵢ≈F(L₁ᵢ, L₂ᵢ) where L₁ᵢ is the brightness line of the first artificial or natural stationary surface (one of 140, 230, 330, 430, 640) in the image, which is preferably ultrasound image of said passive reflector and L₂ᵢ is the brightness line of the said second moving artificial or natural surface (one of 130, 210, 310, 410, 630, 720) in the image, which is preferably ultrasound image of said passive reflector respectively, measured at said time moment tᵢ.

When the system is calibrated, the calculation is based on the utilization of the function P=F (L₁, L₂) derived from previous calibration process: the non-invasive ultrasound measurement with an ultrasound apparatus is provided with further image processing derivation of the variables L₁ and L₂. The further substitution into the formula P=F (L₁, L₂) gives the real time pressure and pressure changes while the series of ultrasound images is recorded. If the system was not previously calibrated, no absolute pressure measurements are provided, but only dynamic pressure changes are calculated/determined from the image series obtained over time.

Based on the above principle the system for the non-invasive ultrasound measurement of the intravascular blood pressure comprises a plurality of passive moving artificial or natural ultrasound beam reflectors optionally implantable or implanted into the cardiovascular system, such as blood vessels or one or both of the heart chambers. Said passive reflectors contain surface elements or surface portions that are stationary or respectively moving under blood pressure changes and adapted to receive and reflect ultrasound beams (or their position in the body be capturable by other image modalities).

The ultrasound beam reflectors may be natural or artificial, the last being integrated or attached to a medical implantable device adapted for delivery and implantation in the body. Such medical implants include for instance stents including self-expandable stents, or occluders such as atrial septal occluders, ventricular septal occluders, (left) atrial appendage occluders, PDA occluders, vascular occluders, vascular plugs, flow regulators, Atrial Flow Regulators (AFR), Aorto-Pulmonary Flow Regulators (APFR), pacemakers, etc.

The system further comprises in an example an ultrasound apparatus adapted to send ultrasound signals to the natural or artificial implanted ultrasound beam reflectors and to receive reflected signals in return, for performing the intravascular pressure measurements.

In general, the system comprises one or more of the following units:
a) A calibration unit containing
   a. at least one catheter based blood pressure sensor, preferably with digital output, permitting to stream the output data into the information system with a processing and/or control unit estimating the volumes of oscillating traceable regions for pressure calculation as the function of the respective volumes, preferably a computer
   b. at least one ultrasound probe (or alternatively or in addition a different image modality) as described below in item b) with (digital) output permitting to stream the output data into the computer/information receiving/processing/storage unit
   c. an information processing unit synchronising input channels and calibrating the pressure calculating model
b) A measurement and calculation unit containing
c) at least one ultrasound probe with at least one transducer configured to convert an electro-magnetic input or control signal into a mechanical ultrasound signal to be transmitted towards a surface, and configured for the reverse conversion of reflected or echoed, incoming mechanical ultrasound signals into electro-magnetic measurement signals, wherein said transducer transmits the direct output ultrasound signal and receives the reflected ultrasound signals;
d) at least one beam former unit configured to provide a desired shape of the electro-magnetic signal in the transmission mode;
e) at least one transmitter unit generating the electro-magnetic signals with their further transformation into ultrasound signals by the transducer;
f) at least one receiver unit for the echoed signals;
g) at least one receiver unit for the information signal (or image) processing preparing the variables for the pre-calibrated pressure function and calculating the real time pressure;
h) at least one unit for information data storage; and
i) at least one control unit configured for estimating the volumes of oscillating traceable regions.

The unit for the information processing includes preferably a software, and alternatively or in addition a hardware which is for instance comprised of:
a. an ultrasound apparatus, such as disclosed above, and preferably with a communication interface such as a wireless communication unit and/or USB port capability;
b. a client device such as a smartphone/tablet/personal computer with a user interface and a client application installed; optionally integral part of the ultrasound apparatus (a) and/or in communication thereto;
c. optionally a local medical centre data server; and
d. optionally a cloud information storage unit.

The software system includes code segments for performing an intra body pressure determination or estimation based on at least an image series over time of a region of interest where the pressure is to be determined or estimated in the body from images obtained remote from the region of interest. Preferably the software and/or system is in use operating as follows:
e. Connect the ultrasound apparatus (a) to a client device (b) via suitable communication interface, such as WiFi/Bluetooth/USB, cable;
f. Set the transducer into operation; the user interface, such as a graphical user interface (GUI) including an on-screen image is displayed on a display, preferably of the ultrasound apparatus or a display connected thereto, such as the client display. The ultrasound apparatus is in a first operation mode run in B-mode. For instance the client (b) starts the apparatus to operate in B-mode. A picture formed by the signal is displayed.
g. The transducer is pointed to the region where the reflector for pressure measurement is located inside the body, such as the heart, and hold. Optionally the signal direction is adjusted according to the displayed image until the reflective implanted membrane is visible on the image.
h. When the membrane or the region of interest is identified in the image data, the ultrasound apparatus is switched to a second mode of operation, the M-mode, or the generalized M-mode, being the set of M-modes corresponding to all formed beams. For instance, the client application (b) provides for the identification. The membrane may be automatically recognized by suitable image processing steps, and switched to M-mode, or the generalized M-mode. The transducer is then retrieving the signal changes for a certain time length, preferably for a number of seconds. Pressure inside the body is then calculated by the control unit based on the analysis of the accumulated M-modes.
i. Upon the successful retrieval, which can be confirmed by suitable software steps in a control unit, the results may be displayed and/or further processed. This may be done by the client application (b). In addition, the ultrasound apparatus may be returned to first operational mode, the B-mode.
j. The measurement results including intra-body pressure values may then be manually or automatically uploaded to a local medical centre server (c), a cloud information storage (d), and/or stored otherwise, e.g. in a memory of the client device.

The current approach is based on a combination of both B-mode and the generalized M-mode imaging.

B-Mode or 2D mode or brightness mode in ultrasound scanning, is a cross-sectional image representing body components through the simultaneous scanning by a linear array of transducers and represented as a two-dimensional image on a screen.

M-mode or TM-mode is or motion mode in ultrasound scanning, permits to fix a specific scan line in B-Mode and simultaneously generate its real time evolution as a vector time series. Current approach is based on the simultaneous analysis of the set of M-modes corresponding to all formed beams (generalized M-mode imaging).

D-mode or Doppler mode in ultrasound scanning makes use of the Doppler effect in measuring and visualizing blood flow or tissue movements in a given sample volume.

We call herein the generalized M-mode. it is defined a set of M-modes corresponding to all ultrasound channels at a certain time moment. The B-mode is got from this mode by passing to polar coordinates and respective interpolations. Thus, our analysis is based on the initially acquired data which permits to get the better analysis of all ultrasound channels without B-mode smoothings and interpolations.

An example of an implantation medical procedure for deployment of a passive ultrasound beam reflector inside the body such as inside the cardiovascular system e.g. an appropriate heart region, left and/or right atrium of the heart, or pulmonary artery, comprises
(a) joint deployment of a carrier unit including a passive ultrasound beam reflector and catheter based blood pressure sensors inside a catheter sheath, such as being releasably attached, preferably at a proximal end, to a connector, such as a capturing unit, such as a claw, being arranged at a distal end of a delivery unit, such as a guide wire or pusher wire of interventional cardiology, configured for releasable attaching said passive ultrasound beam reflector to said connector;
(b) transvascular transportation of the carrier unit to a cardiovascular location, such as a heart region, through the inside of said sheath by means of guide wire or pusher wire manipulations, e.g. by pushing the carrier unit including the passive ultrasound beam reflector through the sheath towards its distally open end;
(c) orientation and deployment of the said carrier unit by means of said guide wire or pusher wire manipulations at said cardiovascular location, such as inside the heart, preferably guided based on fiducial markers on the capturing unit and/or said delivery unit, such as being visible on ultra-sound or fluoroscopy equipment;
(d) anchoring of the said carrier unit and/or passive ultrasound beam reflector into tissue at said cardiovascular location, such as the heart muscle tissue or a blood vessel wall tissue by means of a tissue anchoring unit, such as at least one screw, hook, spring, flange, or the like, at the carrier unit or passive ultrasound beam reflector, or alternatively or in addition based on shape memory material and characteristics thereof to allow fixation;
(e) releasing said carrier unit from the said capturing unit of the delivery unit;
(f) simultaneous calibration recordings of the pressure from catheter based blood pressure sensors and imaging unit to achieve the calibration parameters;
(g) extracting of the sheath from the heart region and the body.

The carrier unit may be a medical implantable device as mentioned above.

Releasable attachment may be made in any suitable form, such as threaded screw attachment, gripper, forceps, thermal release attachment, etc.

The system, method, software and use of the present dislcosure permits to directly measure a blood pressure through a passive sensor implanted into an artery or heart itself.

Pressure values such determined may provide valuable diagnostic information for potential therapeutical treatment of a patient.

### BRIEF DESCRIPTION OF THE DRAWINGS

These and other aspects, features and advantages of which embodiments of the invention are capable of, will be apparent and elucidated from the following description of embodiments of the present invention, reference being made to the accompanying drawings, in which

FIG. 1 depicts a schematic illustration of two versions of a standalone passive ultrasound beam reflector (ball tipped narrow reflector and membrane type reflector) with the ultrasound beam reflecting surfaces deployed in a blood vessel;

FIG. 2 depicts a schematic illustration of a medical implantable device, in an example of an Atrial Flow Regulator (AFR) or APFR (Aorto-Pulmonary Flow Regulator), see Patent Application WO 2016 038115, which is incorporated herein by reference in its entirety for all purposes, namely a passive ultrasound beam reflector, here in form of a membrane with the ultrasound beam reflecting surfaces based measuring the blood pressure in the Right Atrium (the capturing unit resides in the right blood circle); APFR does not significantly differ from AFR device in its geometry, though differs significantly in the method of implantation see Guo K, Langleben D, Afilalo J, Shimony A, Leask R, Marelli A, Martucci G, Therrien J. Anatomical considerations for the development of a new transcatheter aortopulmonary shunt device in patients with severe pulmonary arterial hypertension. Pulm Circ. 2013 September; 3(3):639-46. doi: 10.1086/674328. Epub 2013 Nov. 18, which is incorporated herein by reference in its entirety for all purposes;

FIG. 3 depicts a schematic illustration of the Atrial Flow Regulator (AFR) or APFR (Aorto-Pulmonary Flow Regulator) based passive ultrasound beam reflector with the ultrasound beam reflecting surfaces, deployed in the Left Atrium (the capturing unit resides in the right blood circle);

FIG. 4 depicts a schematic illustration of an Atrial Flow Regulator (AFR) based passive ultrasound beam reflector with the ultrasound beam reflecting surfaces deployed both in the Left and Right Atrium (the capturing unit resides in the right blood circle);

FIG. 5 depicts a schematic illustration of a blood pressure measuring, registration and reporting medical information system;

FIG. 6 depicts a schematic illustration of Ultrasound Transducer interaction with the passive membrane inside the blood vessel or the heart;

FIG. 7 depicts a schematic illustration of another 3d visible via Ultrasound Transducer example of both the standalone passive ultrasound beam reflector being attached to a Atrial Flow Regulator (AFR) or APFR (Aorto-Pulmonary Flow Regulator);

FIG. 8 depicts a flow chart illustrating an implantation medical procedure;

FIG. 9 depicts a schematic illustration of a passive ultrasound beam reflector being attached as positioned at a distal end of a self-expandable stent, incorporated into an Atrial Flow Regulator (AFR) or APFR (Aorto-Pulmonary Flow Regulator). The reflector is attached to the stent with the help of additional ball acting as immobilizer of the stent relatively to AFR/APFR.

FIG. 10 depicts a schematic illustration of a passive ultrasound beam ball tipped narrow reflector being attached as positioned at a distal end of a self-expandable stent, incorporated into an Atrial Flow Regulator (AFR) or APFR (Aorto-Pulmonary Flow Regulator). The reflector is attached to the stent with the help of additional ball acting as immobilizer of the stent relatively to AFR/APFR and serves mainly for Left Atrial Pressure measurements.

FIG. 11 depicts a schematic illustration of a passive ultrasound beam ball tipped narrow reflector being attached as positioned at a proximal end of a self-expandable stent, incorporated into an Atrial Flow Regulator (AFR) or APFR (Aorto-Pulmonary Flow Regulator). The reflector is attached to the stent with the help of additional ball acting as immobilizer of the stent relatively to AFR/APFR and serves mainly for Right Atrial Pressure measurements.

FIG. 12 depicts a schematic illustration of a blood pressure calibration and measuring system (1100), where the pressure sensors (1200) are connected with the pressure monitor (1400) which in case of analogue output is connected through an oscilloscope (1300) with a computer.

FIG. 13 depicts the generalised M-Mode of ultrasound image as received in polar coordinates (2000) with the selected region of interest image (2100) compression to the mean value (2200) resulting in the depth brightness column (2300);

FIG. 14 depicts the correspondence of the objects in the region of interest to the anatomical objects as ultrasound signature of the top of the image (2500), chamber wall (2600), membrane ball position (2700), AFR/inter-atrial septum (2800);

FIG. 15 depicts the images sequence (3000) compressed into the union of the depth brightness lines (3100) forming a new compressed image showing the brightness changes over the time (photometry);

FIG. 16 compares the result of the photometry (compressed images over the time) with the micro-manometer catheter based pressure meter synchronized results;

FIG. 17 depicts the new compressed image (from FIG. 15) interior structure as the surface in 3D space, which is rotated in the lower image;

FIG. 18 depicts the search regions for the model parameters L₁, L₂, . . . in the compressed image (from FIG. 15), such as the chamber wall (3200), membrane ball position (3300), AFR/inter-atrial septum (3400);

FIG. 19 depicts the found model parameters L₁, L₂, . . . in the compressed image (from FIG. 15), such as local ridges (3500) of the chamber wall/upper path (3200) and AFR/lower path (3400);

FIG. 20 depicts the found model parameters L₁ₜ, L₂ₜ, . . . , in example, corresponding to chamber wall/upper path (3200) and AFR/lower path (3400) fitting the regression model (3600) in the compressed image (from FIG. 15);

FIG. 21 depicts the calculation results of the fitted regression model (red graph) and the micro-manometer catheter based pressure (blue graph) with 2 parameter model corresponding to upper and lower paths from FIG. 20;

FIG. 22 depicts the basis for the model improvement using the brightness changes (3700) in the surface of FIG. 17;

FIG. 23 depicts the brightness accounting 3 parameter model (red graph) calculations against the simple 2 parameter model (blue graph);

FIG. 24 depicts the overall system testing results where the upper graph shows the calibration results of the ultrasound image processing algorithm to the micro-manometer catheter based pressure data and the second shows the model calculation reproducing the other series of the micro-manometer catheter based pressure data. It is clearly seen that the most correlated normalized brightness line has considerably improved the very small details of the micro-manometer catheter based pressure (blue graph) and the pressure movements (yellow graph) comparing to 2 parameter model (red graph);

FIG. 25 is a schematic illustration of a PFO Occluder Example (Occlutech Funnel Occluder, single distal disc layer (5000), central channel (5100), one clamp (5200)) WO2005020822A1;

FIG. 26 is a schematic illustration of ASD Occluder Examples (Occlutech double disc occluder, WO07110195 left), (WO 1997/042878 right);

FIG. 27 is a schematic illustration of LAA Occluder Examples (WO2007054116A1 left) (WO2013060855A1 right);

FIG. 28 is a schematic illustration of a Mitral valve replacement and/or annuloplasty structure Example WO2012127309(A1);

FIG. 29 is a schematic illustration of a Para valvular Leakage Device Example (WO2013041721A1);

FIG. 30 is a schematic illustration of an example of a medical implant (6000) when implanted at the inter-atrial region (6100); and

FIG. 31 is a schematic illustration of medical implants for occlusion of PFO and/or ASD (WO2010104493(A1) left and WO2010151510(A1) right.

### DESCRIPTION OF EMBODIMENTS

Specific embodiments or examples of the invention will now be described with reference to the accompanying drawings. This invention may, however, be embodied in many different forms and should not be construed as limited to the embodiments demonstrated herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers may refer to like elements.

The present description of the current invention is given with reference to a blood vessel or a heart chamber as an example only. It should be born in mind however that the present invention is not limited strictly to a blood vessel or a heart chamber, but can be easily adapted to any medium transparent for ultrasound or other waves with the need to measure pressure changes of the liquid flow. Examples include one or more passive ultrasound beam reflectors positioned in the lymphatic system, bile ducts, urinary ducts, subarachnoid space around the brain and spinal cord (Cerebrospinal fluid), inside or exterior of the lung in the chest wall, etc. for measuring pressures and dynamic progress thereof. Correspondingly, implants adapted for implantation in such a location in the body having optionally at least one attached passive ultrasound beam reflectors are provided.

Alternatively or in addition to ultrasound to generate series of images to be analysed for the intra body pressure determination, for instance Echo Doppler, Magnetic Resonance Imaging (MRI), or ionizing radiation based imaging systems like Roentgen (X-Ray, Computer tomographic Imaging [CT]) can be provided as medical imaging modalities for generating the input for the pressure determination.

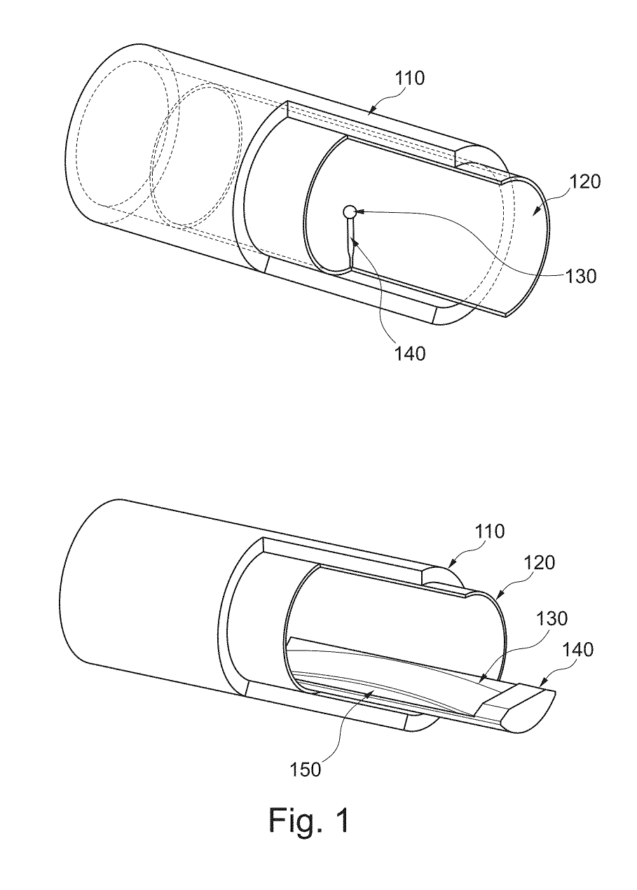

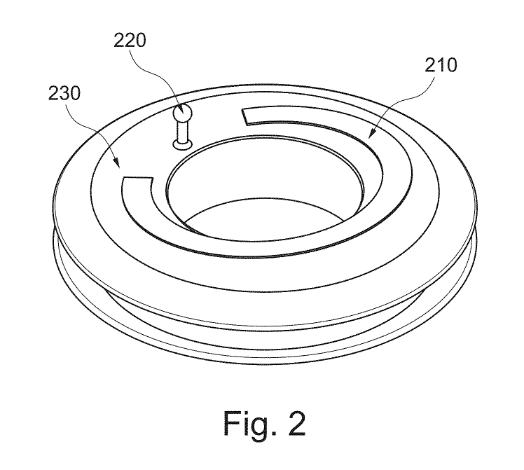

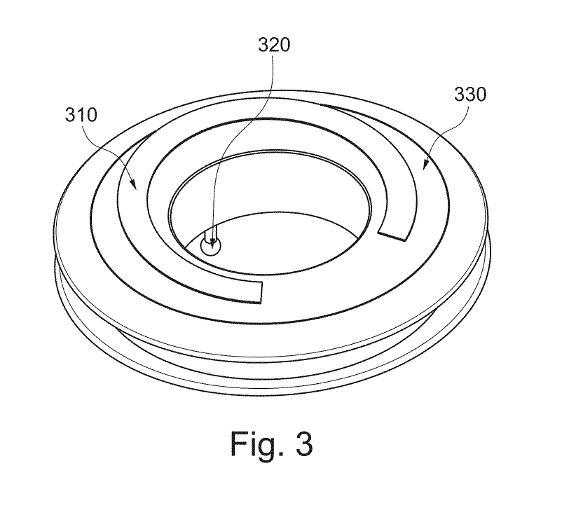

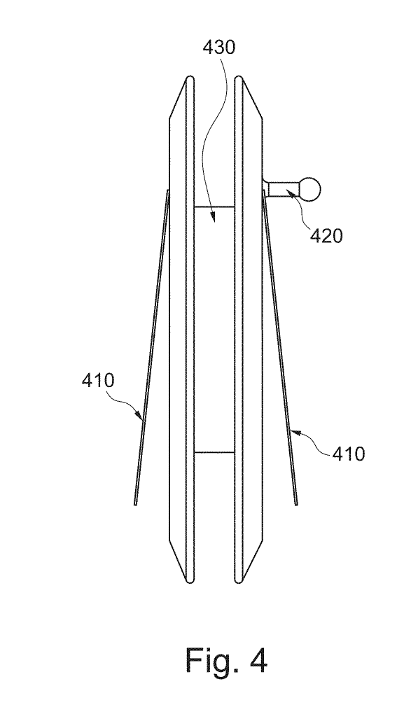

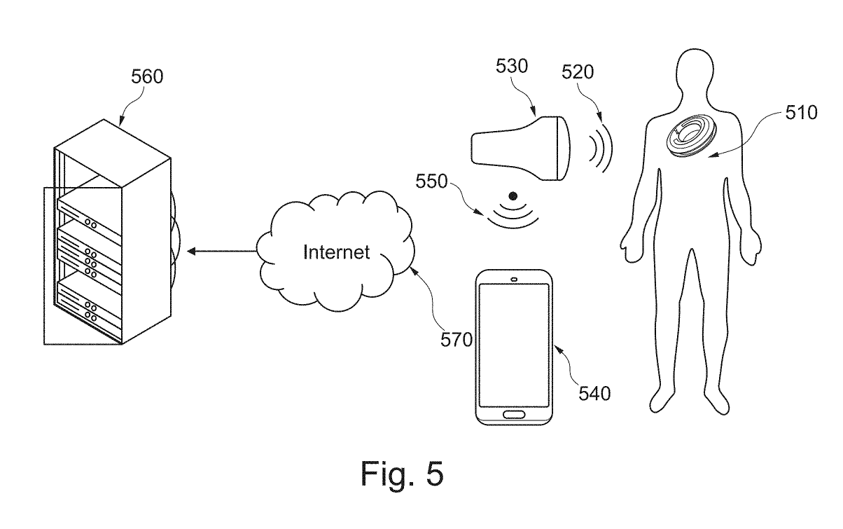

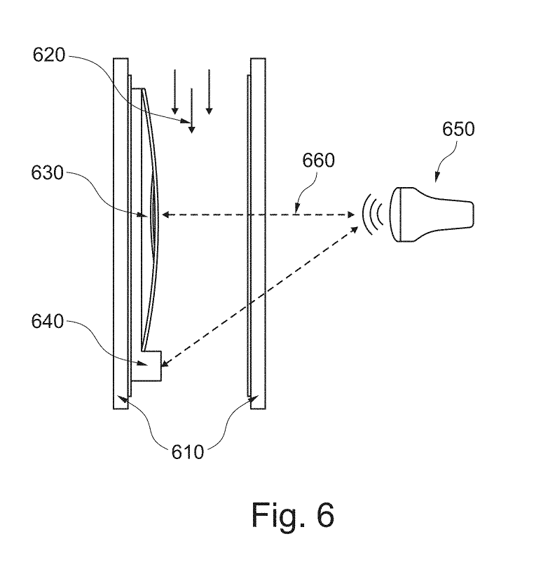

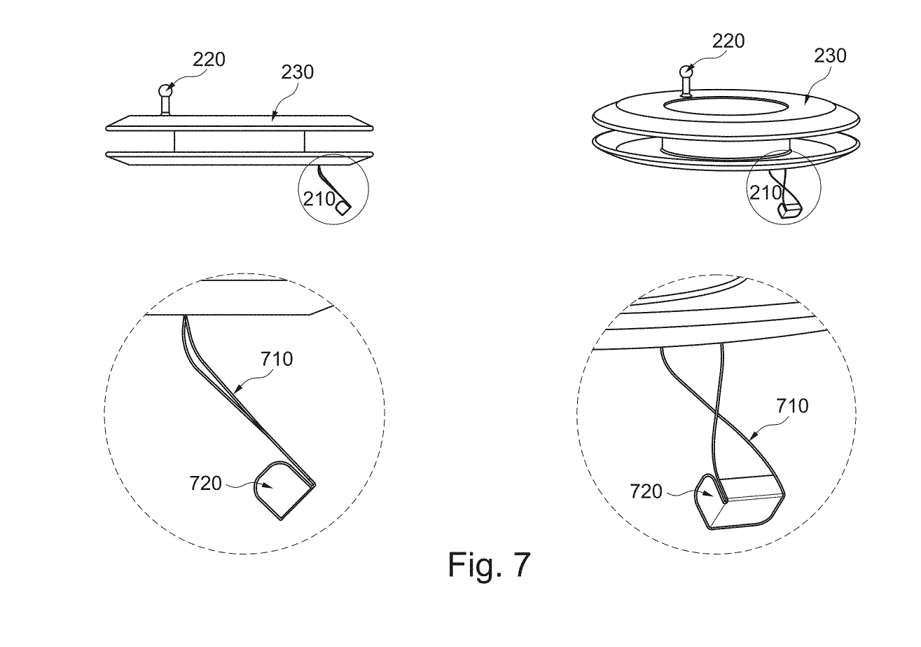

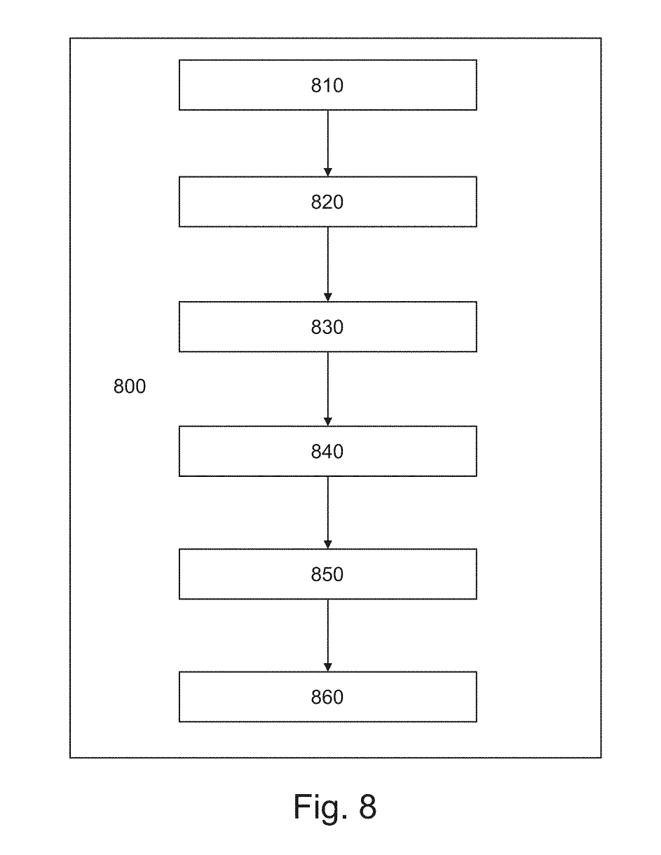

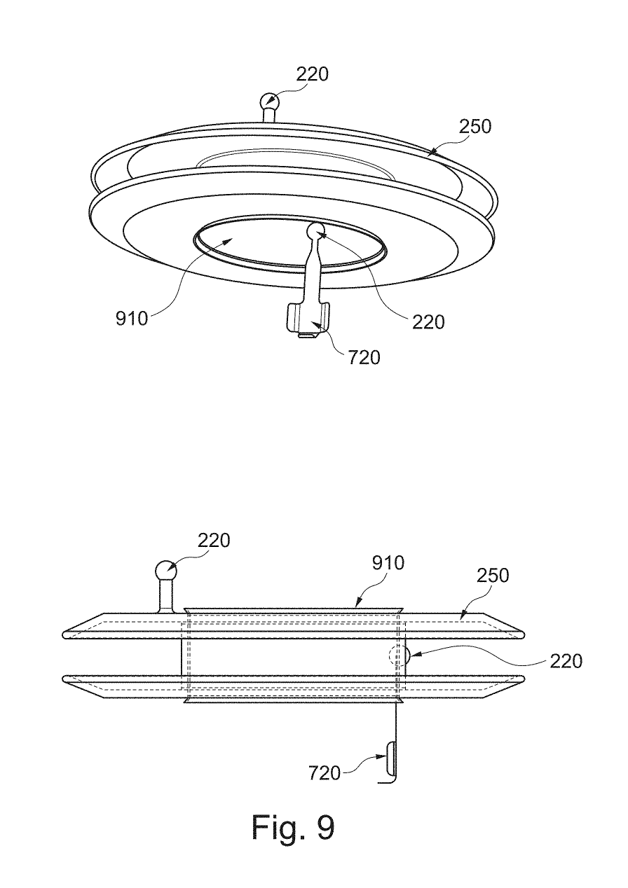

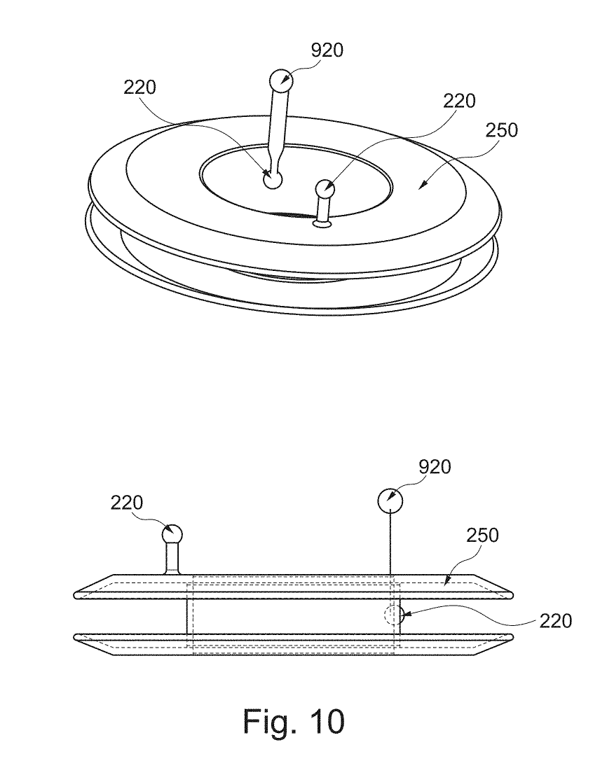

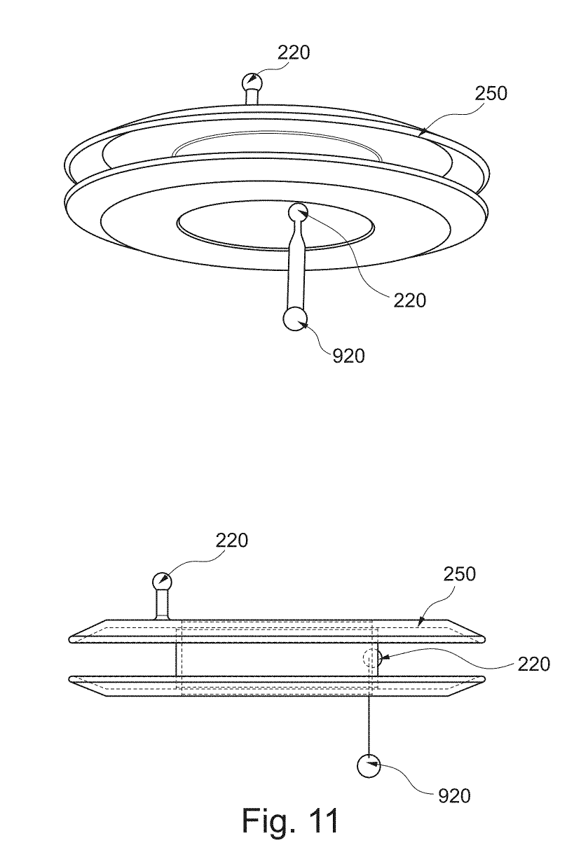

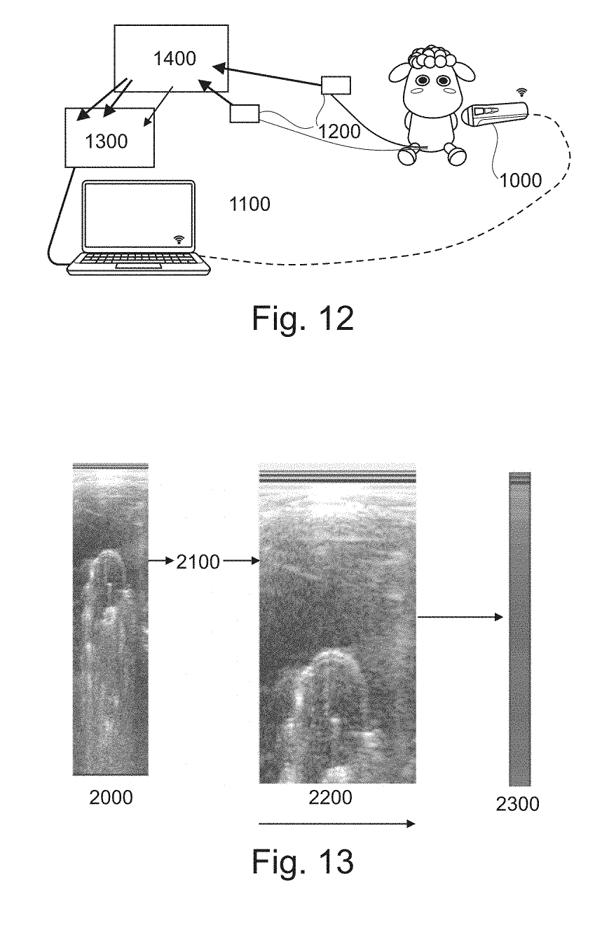

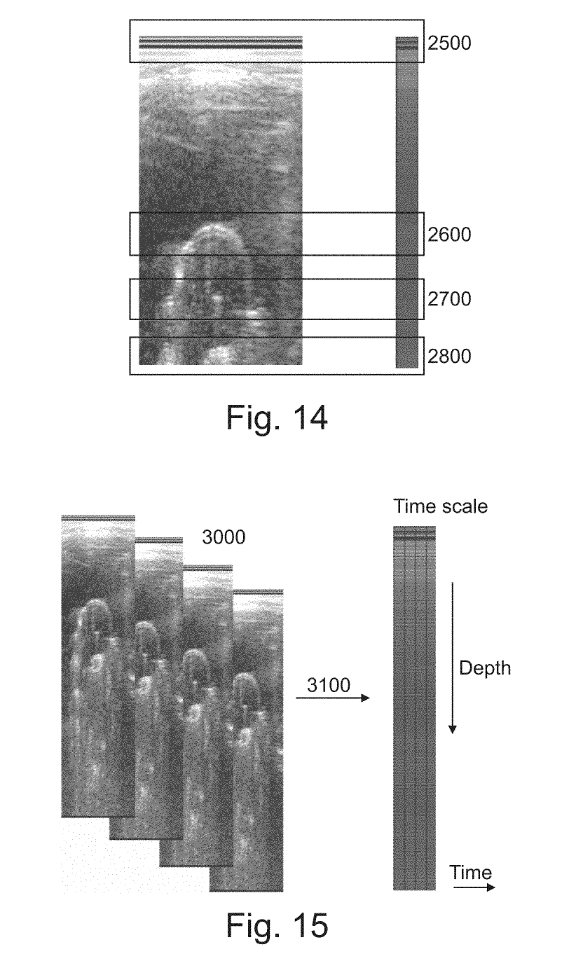

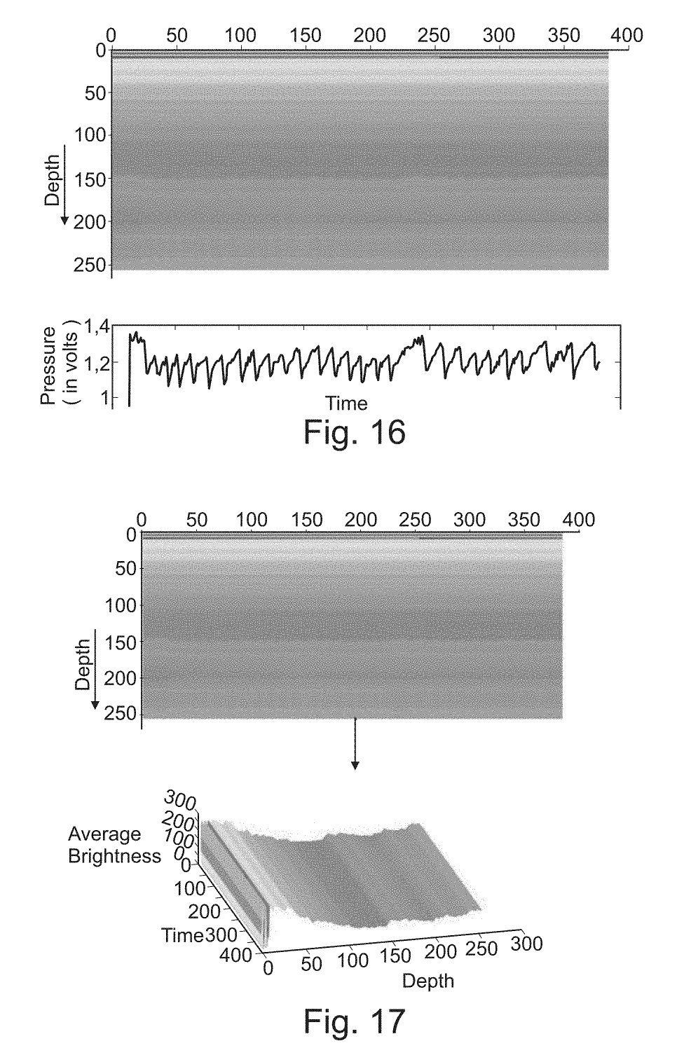

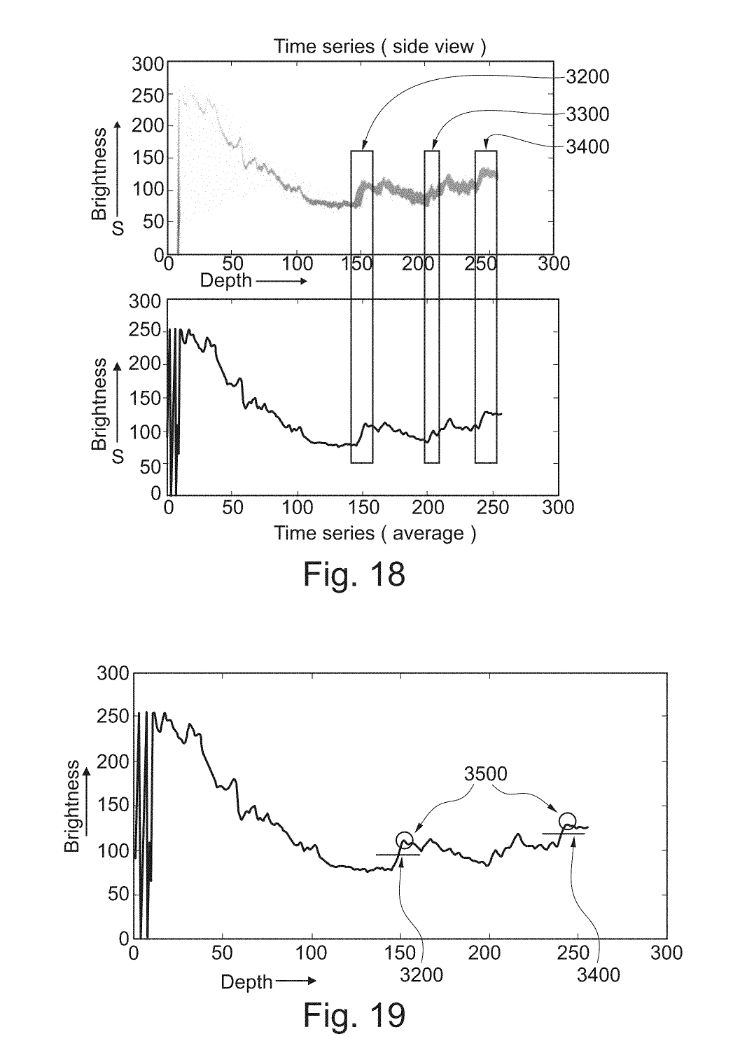

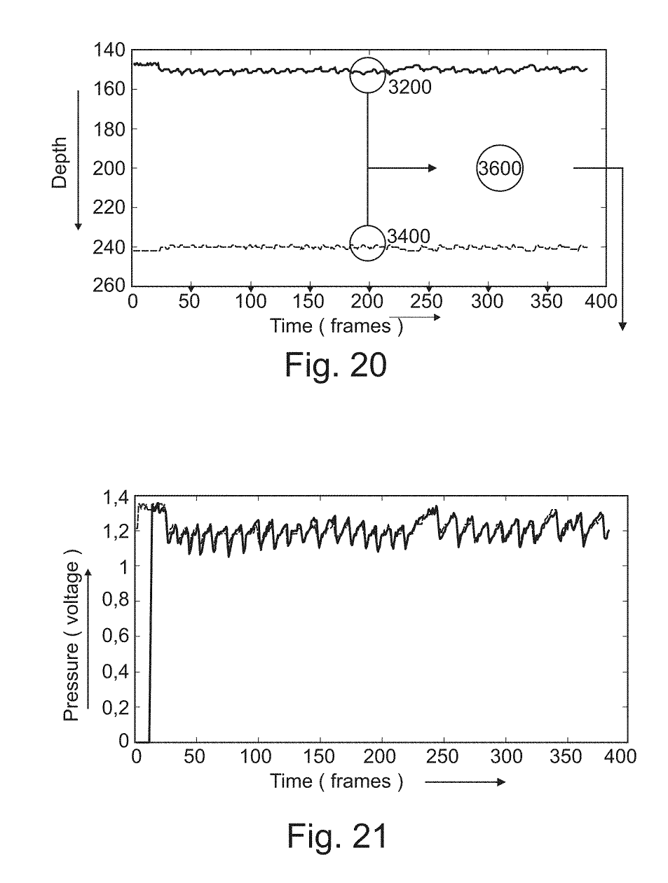

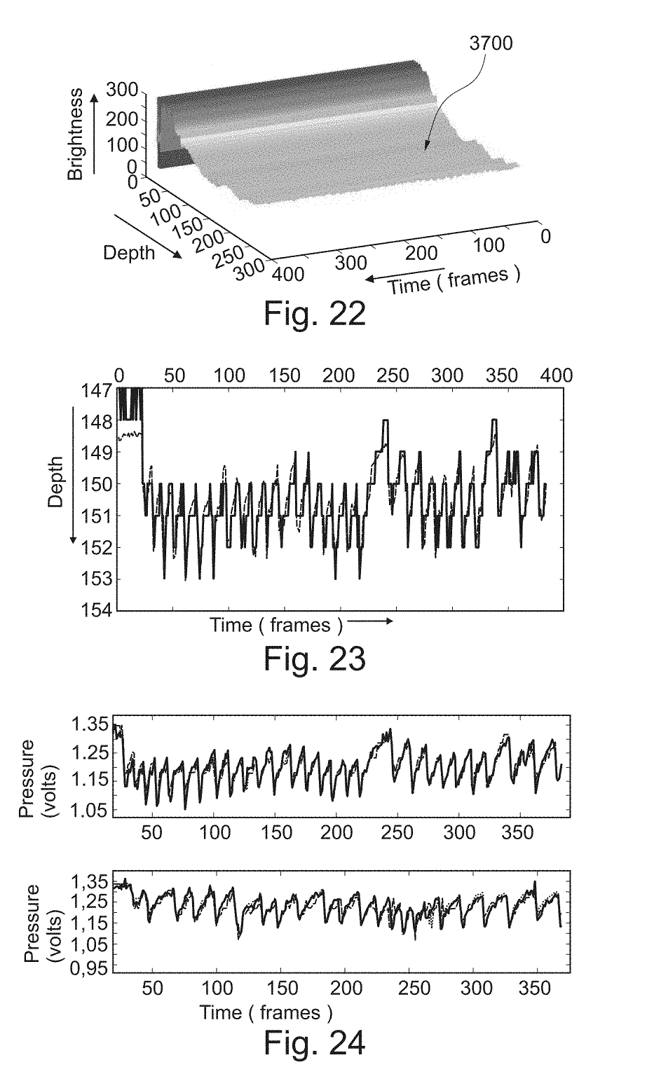

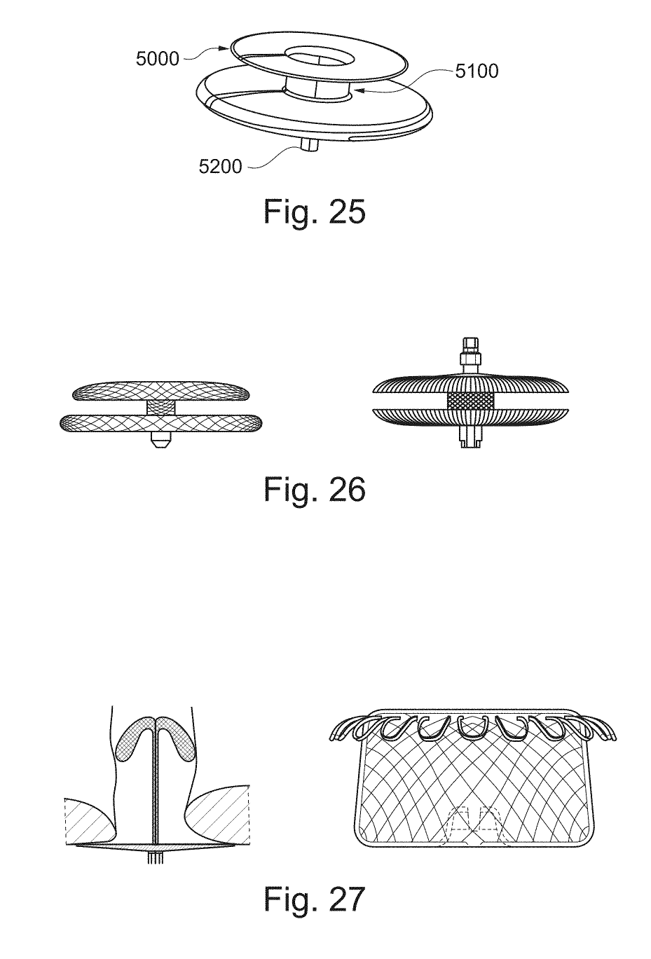

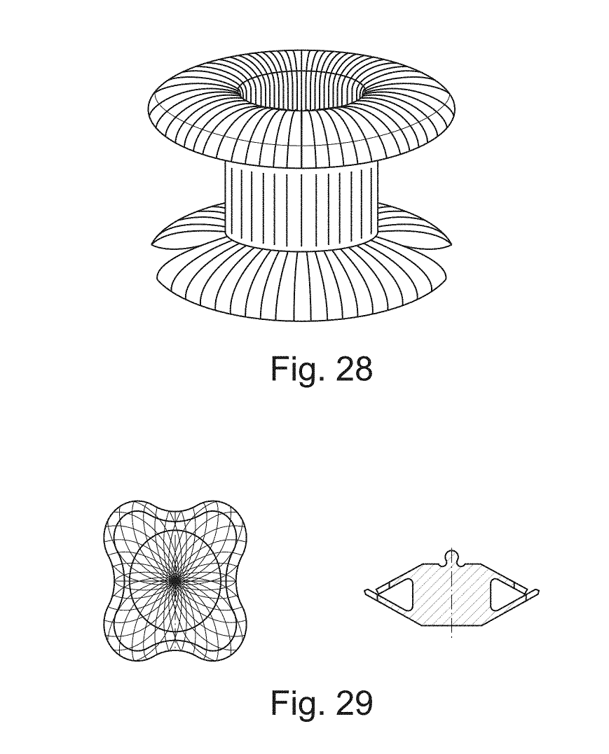

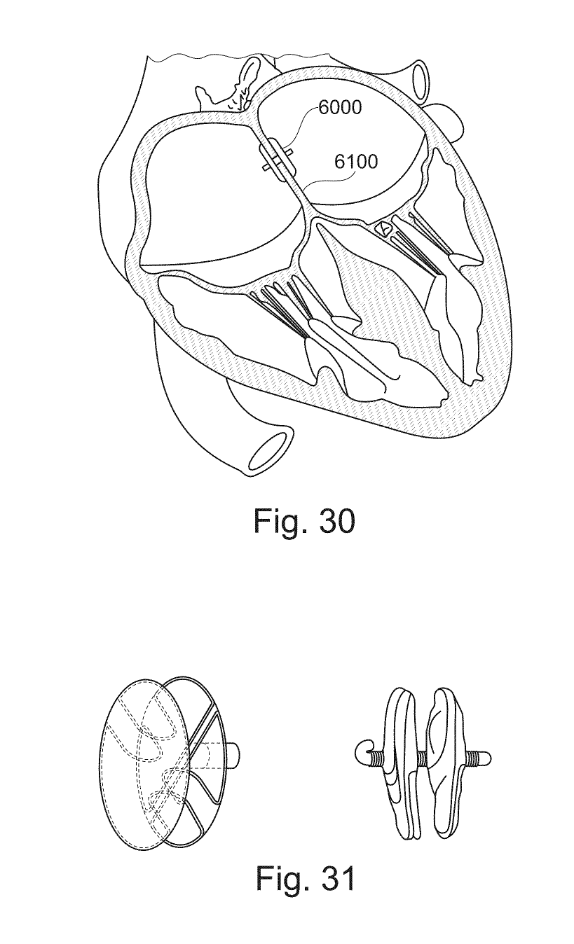

In accordance with preferred embodiments a system comprises for example:
1. One or more artificially implanted or natural passive ultrasound beam reflectors implantable or implanted into the body, such as into the cardiovascular system of a mammal, such as in one or more blood vessels or heart chambers. The reflectors comprise two kinds of surface elements arranged relative each other. Firstly, a stationary natural, or implanted (substantially not moving under blood pressure, i.e. a reference reflector surface) surface element adapted to receive and reflect ultrasound beams. Secondly a natural, or implanted surface element configured to move under (blood when implanted in the cardiovascular system) pressure changes and adapted to receive and reflect ultrasound or other beams. The beam reflectors hence include a first fixed surface at a constant position independent from the (intravascular blood) pressure changes at its implantation location. Further, a second moving surface is included in the ultrasound reflector, the second surface adapted to oscillate under (blood) or in line with (blood) pressure changes at the implantation site, such as inside a blood vessel or a location of the heart. Preferably, the passive ultrasound beam reflector(s) are provided arranged at a medical implant to be implanted at a location inside the body where pressure changes may occur, as for instance described above.
   Preferably, the implanted passive ultrasound beam reflector (or the medical implant with attached passive ultrasound beam reflector(s)) is at least partly endothelialized (overgrown with tissue) after a certain implantation time. In this case, a thin tissue layer will not hinder the movable membrane from oscillation with the pressure of the adjacent fluid (blood) across the tissue layer.
   Alternatively, or in addition the surfaces in blood contact can be suitable coated (e.g. heparin coated or with another pharmaceutical substance as desired) and/or being made of a material compatible with blood.
   The implanted passive ultrasound beam reflectors beams have suitable shape and sizes for being collapsible into a catheter for delivery, for being attachable to a carrier, and/or for fitting with reliable anchoring at the implantation size. Sizes can be as small as a few mm. The beams can be foldable and be of a resilient material returning to their expanded, substantially planar relaxed shape as shown in the FIGS. Shapes include rectangular, square, circular, semi-circular, oval, open oval, generally elongate, or the like.
   Some exemplary embodiments will now be described. It should be noted that the exemplary embodiments should not be taken as isolated from each other, but features of embodiments may be present in other embodiments, even if not explicitly described or shown in the FIGS. For instance, the open ring shaped reflector of FIG. 2 could be in the form of a standalone reflector device, or attached to other medical implants than an AFR. Moreover, rectangular membranes, or a ball tipped narrow reflectors (920) as shown in FIG. 1, could be attached to medical implants, like the AFR in FIG. 2, FIG. 9-11 but also alternatively or in addition to other exemplary medical implants, such as those are shown in FIG. 25 to FIG. 31. These devices may be provided with or with suitable surfaces for identification in images for the herein described pressure determination when implanted. For instance in FIG. 25 a schematic illustration of a PFO Occluder Example (Occlutech Funnel Occluder, single distal disc layer, central channel, one clamp). The device is disclosed without the present improvements described herein in WO2005020822A1, which is incorporated herein by reference in its entirety for all purposes. Particular reference is taken to the device shown in the Figure and the related description therein.
      In FIG. 26 a schematic illustration is provided of ASD Occluder Examples (Occlutech double disc occluder, WO07110195 left), (WO 1997/042878 right) without the present improvements described herein; Both WO07110195 and 042878 are incorporated herein by reference in its entirety for all purposes.
   In FIG. 27 a schematic illustration of LAA Occluder Examples is provided (WO2007054116A1 left) (WO2013060855A1 right) without the present improvements described herein; WO2007054116A1 and WO2013060855A1 are incorporated herein by reference in its entirety for all purposes.
   In FIG. 28 a schematic illustration of a Mitral valve replacement and/or annuloplasty structrure Example is provided (WO2012127309(A1)) without the present improvements described herein; WO2012127309(A1) is incorporated herein by reference in its entirety for all purposes.
   In FIG. 29 a schematic illustration of a Paravalvular Leakage Device Example is provided. (WO2013041721A1) without the present improvements described herein. WO2013041721A1 incorporated herein by reference in its entirety for all purposes.
   FIG. 30 is a schematic illustration of an example of a medical implant when implanted at the atrial region.
   FIG. 31 is a schematic illustration of medical implants for occlusion of PFO and/or ASD (WO2010104493(A1) left and WO2010151510(A1) right; which are incorporated herein by reference in its entirety for all purposes).
   The medical implants are merely examples of such implant, but all facilitate an advantageous pressure determination of a pressure at the implantation region, wherein the pressure is determined from images of a medical imaging modality in accordance with systems, methods, software as described herein. In this manner, an oscillation (such as with cyclic blood pressure as actuated by the cardiac pumping cycle) at the region of implant is advantageously determinable from a medical image series over time. The oscillation is preferably relatable to a volume change at the implantation region. The (cyclic) change is determinable from the medical image series. The volume change is in turn correlatable to a pressure change at the implantation region, e.g. in a cardiac atrium, or a pressure for each atrium, a pressure difference between the atria etc, Such pressure are thus advantageously determinable. The pressure may be determined from the implants position themselves. Alternatively or in addition, the pressure may be determined from surfaces of structures attached or integral part of the implants (e.g. membranes). Alternatively or in addition, the pressure may be determined from surfaces of anatomical structures at the region of the implant; either by the anatomical structures and related volume changes themselves, or preferably by means of such one or more implants when implanted at the region (with improved motion identification/detection, or as a reference point, in the medical image series).
   a) In accordance with a first example, a passive ultrasound beam reflector 150 (FIG. 1) is provided in the exemplary form of a plate or contoured membrane which is deformed under the (blood) pressure changes at its location. The device 150 is shown deployed inside a blood vessel, such as the pulmonary artery. In the example, the device is a stand-alone device, i.e. not attached to a carrier medical implant. In similar examples, it may be attached to a medical implant carrier, such as a stent. The reflector 150 includes both stationary 140 and movable 130 surface elements, wherein the movable surface element 130 is moving under (blood) pressure changes when implanted. The moveable surface element is attached at its beam ends to a carrier and/or the stationary surface element 140. Thus an oscillatable beam with a free apex is provided that is deflectable by the pressure at its implantation location. The reflector is shown inside the delivery sheath 120 inside the vessel, here the pulmonary artery 110 during transvascular deployment and before release and anchoring in the vessel. Anchoring of the device 150 may be done in many suitable anchoring means implementing ways, such as with hooks, bows, screws, tissue adhesives, etc. The apex is preferably also present in other examples described herein. The stationary surface element may be arranged in parallel, above or below the oscillatable beam for reflection in substantially the same direction as the oscillatable beam. Alternatively, or in addition, such a stationary surface may be provided adjacent the oscillatable beam as e.g. shown in FIG. 1 (such as the elevated heel shown at an end of the reflecting device).
   b) In accordance with another example, related to the first example, the passive ultrasound beam reflector 210 (FIG. 2) in the form of a plate or contour membrane has an open ring shape. An apex as in the previous example may be provided. As mentioned afore, a moveable membrane 210 is deformed under the blood pressure changes. The reflector 210 is attached to an AFR device playing itself the role of the first fixed surface 230 and having two flanges to be positioned on two different sides of the heart across a septal shunt. The AFR device allows a blood flow through its central passageway as shown. The flanges (here disk formed) retain AFR device at the septal wall in its position. It usually is overgrown with endothelia a certain time after implantation. The moveable reflector part is attached to the proximal end on the surface of the AFR where in the example a delivery connector or capturing unit 220 is positioned on the AFR (Atrial Flow Regulator) device 230. when the AFR is implanted, the aggregate of AFR and reflector 210 allows for a controlled shunt between left and right atria. The reflector 210 provides for pressure measurement on the proximal side of the AFR. Being able to measure pressure at an AFR is a long felt need and provided in an advantageous way by these examples having an integrated pressure measuring reflector 210 with an AFR. Pressure is an important parameter to determine the effective implantation of an AFR as the shunt is created to treat a hypertonic condition by providing a desired shunt flow between the right and left heart. Alternatively, or in addition the reflector 210 may be attached to or integrated with other medical implants than AFR devices, such as medical implants of the occlusion devices type including Atrial Septal Occluders, Ventricular Septal Occluders, stents, etc.
   c) In accordance with another example, related to the other examples herein, the passive ultrasound beam reflector 310 (FIG. 3) is provided in the form of a plate or contoured membrane in the form of an open ring. The reflector 310 is deformed under the blood pressure changes and is attached to the distal end surface of an AFR, i.e. on the surface opposite to the delivery connector or capturing unit 320 of the AFR (Atrial Flow Regulator) device 330, when deployed to create a shunt between left and right ventricles as described above.
   d) In accordance with another example, related to the other examples herein, two passive ultrasound beam reflectors 410 (FIG. 4) in the form of a plate or contoured membrane deformed under the blood pressure changes are attached to both the distal and proximal end of an AFR (Atrial Flow Regulator) device 430 deployed to create a shunt between left and right ventricles. It can be seen that the reflectors 410 moveable surface portion, as shown in FIG. 2 or 3, of open ring shape protrude at their apex from the AFR device and are attached at their ring ends only to provide an increased oscillation. The illustrated dimension of the protrusion may be smaller in order to not hinder endothelisation. A ring may be closed as an alternative and attached at a perimeter only allowing a certain protrusion and movement of a ring portion. Also, a ring, open or closed as described herein is generally flat to allow for the desired reflectivity and movability/flexibility. Having two reflectors on two sides of the shunt when implanted allows for differential pressure measurements across the shunt when the AFR is implanted. As the shunt is of defined diameter and length, given by the expanded dimension of the AFR, the blood flow across the shunt is determinable.
      Each of the plurality of reflectors may be identified by position and direction of the ultrasound probe towards the reflector, respectively. Alternatively, or in addition, the size and/or shape of various reflectors may be different so that a particular reflector is identifiable for measurement at a particular location in the body, e.g. in the ultrasonic image taken in B-made by suitable image recognition software. Fiducial markers in various patterns may also assist in identifying a particular reflector.
   e) In accordance with yet another example, related to the other examples herein—the passive ultrasound beam reflectors 210, 310, 410 in the forms of a plate or contoured membrane that is deformed under the blood pressure changes are attached to the distal and/or proximal end of an APFR (Aorto-Pulmonary Flow Regulator) device similar to the AFRs 230, 330, 430, deployed to create a shunt between the left pulmonary artery and the descending aorta. Pressure measurements are thus provided at the left pulmonary artery side and/or the descending aorta side of the APFR when implanted.
   f) In accordance with another example, related to the other examples herein, the beam reflector has alternatively, or in addition a passive ultrasound 3 dimensional (in the sense that it has multiple reflective surfaces and is visible by an ultrasound device from any viewing angle) beam reflector 720 (FIG. 7) in the form the contour membrane deformed under the blood pressure changes and shifted directly into the blood flow with the help of the shape memory alloy rods 710, can be attached both to AFR or APFR 230 playing the role of stationary ultrasound reflective surface or perform as stand-alone device in left pulmonary artery being fixed on the walls with the shape memory alloy rods playing the role of the stationary surface. The 3 dimensional ultrasound device detectable body has for instance orthogonally and parallel arranged reflective surfaces, such as shown in the FIG. 7.
   g) In accordance with yet another example, related to the other examples herein, the passive ultrasound beam reflector (720) or a ball tipped narrow reflector (920) from FIG. 9-11 being attached positioned at a distal end of the (self-expandable) stent 910. It is in the shown example incorporated into Atrial Flow Regulator 250 (AFR) or APFR (Aorto-Pulmonary Flow Regulator). The reflector 720 or a ball tipped narrow reflector (920) is for instance attached to the stent 910 with the help of additional ball 220 acting as immobilizer of the stent relatively to the AFR/APFR 250. The self-expandable stent 910 can be substituted by a regular, e.g. balloon expandable, stent, but then the balloon catheterization is needed. This option provides the freedom of using the same product to measure Right Atrium (RA) or Left Atrium (LA) pressure with no need to alter an AFR device. The stent 910 is integrated with the AFR device upon expansion in its inner flow channel. With minor changes this example can be used alternatively to measure Pulmonary Artery (PA) pressure. In this case the passive ultrasound beam reflector 720 is arranged orthogonal to the interior surface of the the self-expandable stent 910 as on FIG. 1.
2) The above described preferred examples of reflectors and/or medical implantable devices, further may be comprised in a system an ultrasound apparatus 530 (FIG. 5) adapted to send ultrasound signals 520 to the one or more natural, or implanted ultrasound beam reflectors 510 and receive reflected signals in return, and performing the measurements/pressure determinations. The system preferably comprises one or more of the following units:
   a) The calibration unit (FIG. 12) containing
      a. at least one catheter based blood pressure sensor (1200) with digital output or Medical pressure monitor with analogue data output combined with oscilloscope digitalizing the output and permitting to stream the output data into the information system, preferably the computer (1100)
      b. at least one ultrasound probe (1000) as described below in item b) with digital output permitting to stream the output data into the computer/information receiving/processing/storage unit
      c. an information processing unit synchronising input channels and calibrating the pressure calculating model
   b) at least one ultrasound probe 530 (FIG. 5), or probe 650 (FIG. 6), with at least one transducer providing the direct conversion of the electro-magnetic signal into mechanical ultrasound signal and the reverse conversion of the mechanical ultrasound signals into electro-magnetic signals, wherein said transducer transmits and receives the direct and reflected ultrasound signals;
   c) at least one beam former unit (not shown) providing the necessary shape of the electro-magnetic signal in the transmission mode;
   d) at least one transmitter unit (not sown) generating the electro-magnetic signals with their further transformation into ultrasound signals by the transducer 530, 650;
   e) at least one receiver (here in probe 530, 650) for the echoed signals;
   f) at least one unit of the information processing device 540 preparing the variables for the pre-calibrated pressure function and calculating the real time pressure;
   g) at least one unit of the information storage 540, 560 and at least one control system also contained in the blocks 540-560 in FIG. 5. The control system can be distributed between the user information processing device 540 and central or medical institution server 560 over the Internet or Intranet 570.
3) The system according to the above eamples further provides the subsequent synchronous recordings of the pressure measurements from alternative pressure meters, such as catheter based blood pressure sensors and imaging, preferably ultrasound measurements with the said probes and the calculation method for the best fit of the measured pressure values Pᵢ at the implantation areas of said reflector and at time moments tᵢ as a function Pᵢ≈F(L₁ᵢ, L₂ᵢ) where L₁ᵢ is the brightness line of the first artificial or natural stationary surface (one of 140, 230, 330, 430, 640) in the image, which is preferably ultrasound image of said passive reflector and L₂ᵢ is the brightness line of the said second moving artificial or natural surface (one of 130, 210, 310, 410, 630, 720) in the image, which is preferably ultrasound image of said passive reflector respectively, measured at said time moment tᵢ.
   The calculation is based on the utilization of the best fitted through calibration function F such that Pᵢ≈F (L₁ᵢ, L₂ᵢ) based on calibration measurements of said dependency Pᵢ from the parameters L₁ᵢ and L₂ᵢ at the varying pressure values in the predetermined range.
   One may not bound oneself by 2 brightness line parameters L₁, L₂ (U (Upper) and L (Lower) in FIG. 19) but pass to pressure function P of a location inside a body, including determining the brightnesses L₁, L₂, . . . , Lₙ of moving or fixed natural or artificial surfaces containing at least one optional passive reflector previously implanted at a implantation area at said location inside said body and at a measurement time t by image processing. The method further includes calculation of a local pressure P at time moments t as a function Pᵢ≈F(L₁, L₂, . . . , Lₙ) of L₁, L₂, . . . , Lₙ based on the calibration fit process, where the function P is being built by the measurements as in at time moments tᵢ of said dependency of Pᵢ from L₁ᵢ, L₂ᵢ, . . . , Lₙᵢ in a predetermined range and for prescribed shape of the function F. For example, F can be a linear function of the brightnesses L₁, L₂, . . . , Lₙ with the coefficients W₁, W₂, . . . , Wₙ optimally fitted for equations Pᵢ≈W₁L₁ᵢ+ . . . +WₙLₙᵢ+C to hold for the subsequent ultrasound recordings (FIG. 21). Said coefficients W₁, W₂, . . . , Wₙ are proportional to the cut areas of the said target volume at given depths orthogonally to the transducer working plane, while the whole sum is approximating the pressure as the function of the target area volume. This follows from the assumption that
   the pressure P (t)=P (V (t)) is the function of the volume of the target region and the volume is approximated according to the Simpson formula as the sum V(tᵢ)=\tilde{W}₁L₁ᵢ+ . . . +\tilde{W}ₙLₙᵢ, where \tilde{W}_{L} are the real cut areas of the said target volume at given depths orthogonally to the transducer working plane and Lⱼᵢ indicate the height of the slices. The procedure of the best fit to the measured data provides the accurate hypothesis for the weights W₁, W₂, . . . , Wₙ.
   Now the algorithm procedure is for example:
      i. To identify the objects of interest as the first fixed surface and the second moving surfaces the ultrasound apparatus 530, 650 is configured to work in 2-Dimensional (2D- or B-) visualization mode, or simply B-mode, see for example, FIG. 5.
4) To measure the brightness lines L₁, L₂ (say U (Upper) and L (Lower) in FIG. 19) the ultrasound apparatus 530, 650 is configured to work in the generalized time motion mode (TM- or M-mode, see FIG. 5). For stable functioning the system further provides the subsequent tracking of the brightness lines L₁ᵢ, L₂ᵢ at the measurement time moments tᵢ adjustable to 3-dimensional movements of the passive ultrasound beam reflector and/or AFR/APFR.
   ii. From each image in the image sequence (FIG. 15) Average each Horizontal line brightnesses of the region of interest turning each picture turns into a column vector of average brightness-compressed images Iᵢ (see, FIG. 13, this process is called photometry in Astronomy, a remote field of the present description).
   iii. These columns time moments tᵢ are combined into a unique frame (FIG. 15) {Iᵢ} from where the brightnesses L₁, L₂, . . . , Lₙ are derived accounting the movements of local extremes (FIGS. 17 and 18) and are representing either the brightness peak path across the joint horizontally averaged ultrasound images Iᵢ (FIG. 17-20), or the most correlated normalized brightness lines to {L₁ᵢ} and respectively {Lₙᵢ} (FIG. 22) in the set {Iᵢ}, where {L₁ᵢ} and {Lₙᵢ} are the upper and lower brightness peak paths across {Iᵢ}.
   iv. As the paths of pixels does represent accurately enough the pressure values (FIG. 23), we locate the line, where the brightness change B_{c} is most correlated to the upper path, and fit the U (FIG. 21) values to it using linear regression model U=a*B_{c}+b model, substituting the actual values of B_{c} we get adjusted brightness B=a*B_{c}+b normalizing the brightness curve to be independent of initial signal strength. This provides better value resolution than the pixel-coordinate (Y-axis below) only approach (FIG. 23).
   v. The overall system testing results are presented in FIG. 24, where the upper graph shows the calibration results of the ultrasound image processing algorithm to the catheter pressure meter data and the second shows the model calculation reproducing the other series of the catheter pressure meter data. It is clearly seen that the most correlated normalized brightness line has improved precision, despite relatively small details of the pressure movements.
5) The software system processing the measurements from previous item 3) in its preferred example is composed of:
   i) ultrasound apparatus 530, 650 with wireless/USB port capability
   ii) smartphone/tablet/personal computer with client application installed 540
   iii) optional local medical centre server 560
   iv) optional cloud information storage 560
6) The software system of the preferred example as a whole is operating as follows:
   i) Connect the ultrasound apparatus 530, 650 to the client device 540 via WiFi/Bluetooth/USB cable 550
   ii) Ensure the transducer 530, 650 is in operation: on-screen image should appear on the ultrasound apparatus monitor (delegated to client device 540). The client 540 starts to operate the ultrasound apparatus in B-mode, displaying the picture formed by the signal.
   iii) Point the transducer 530, 650 to the heart region and hold, adjust the signal direction according to the image until the membrane 630 is visible.
   iv) The client 540 software application automatically recognizes the membrane 630 and switch to the generalized M-mode—being the set of M-modes corresponding to all formed beams, retrieving the signal changes for a number of seconds.
   v) Upon the successful retrieval, the results will be displayed by the client 540 software application
   vi) Results may be manually or automatically uploaded to local medical centre server 560 or cloud information storage 560 and stored on the client device 540.
7) The passive ultrasound beam reflector (one of 210, 310, 410, 640) according to the above examples is further deployed according to medical procedure described below 800 (FIG. 8) inside the appropriate heart region that can be left and/or right atrium of the heart or inside pulmonary artery, said procedure comprising
   (a) optional deployment 810 of the said passive ultrasound beam reflector inside a standard sheath being attached by means of a proximal end (220, 320, 420) to a capturing unit, such as a claw, being arranged at a distal end of a delivery unit, such as a guide wire of a standard sheath of interventional cardiology, for releasable attaching of the said passive ultrasound beam reflector to said capturing unit,
   (b) endovascular transportation 820 of the said carrier unit to an appropriate heart region inside said sheath by means of guide wire manipulations
   (c) orientation 830 of the said carrier unit by means of said guide wire manipulations inside the heart according to fiducial marks on the said capturing unit and said delivery unit, such as visible on ultra-sound or fluoroscopy equipment
   (d) anchoring 840 of the said passive ultrasound beam reflector
      (i) into the arterial septum together with the AFR device, using AFR delivery procedure,
      (ii) into the left pulmonary artery or descending aorta together with the APFR device, using APFR delivery procedure,
      (iii) into the left pulmonary artery as a stand-alone device using shape memory allow wires preventing the reflector to move along the artery.
   (e) releasing 850 said carrier unit from the said capturing unit of the delivery unit
   (f) extracting 860 of the sheath from the heart and the body.

The present invention has been described using a non-limiting detailed description of various embodiments and examples thereof. It should be appreciated that the present invention is not limited by the above-described examples and that one ordinarily skilled in the art can make changes and modifications without deviation from the scope of the invention as will be defined below in the appended claims.

Below are listed some of the modifications, which are within the scope of invention as defined by the appended claims:

1. The invention can be used with the presently available processes of deployment of the described system through a subclavian jugular or cephalic vein.

2. Instead of Nitinol (nickel-titanium alloy) any Shape Memory Alloy with appropriate properties can be used. Alternatively, or in addition, super elastic or elastic material may be used.

3. Furthermore, the invention can be used for development of the system applicable to any medium transparent for ultrasound waves with the need to measure pressure changes of the liquid flow inside.

It should also be appreciated that features disclosed in the foregoing description, and/or in the foregoing drawings and/or following claims both separately and in any combination thereof, be material for realizing the present invention in diverse forms thereof. When used in the following claims, the terms “comprise”, “include”, “have” and their conjugates mean, “including but not limited to”.

The present invention has been described above with reference to specific examples. However, other embodiments than the above described are equally possible within the scope of the invention. Different method steps than those described above, performing the method by hardware or software, may be provided within the scope of the invention. The different features and steps of the invention may be combined in other combinations than those described. The scope of the invention is only limited by the appended patent claims.

1. A system for providing continuous measurement of the pressures inside a body by means of the processing of the series of images generated by an ultrasound or other medical imaging unit, said system based on the image time series processing, having a control unit adapted for calculating said pressures as functions of volumes estimating the volumes of oscillating traceable regions, said regions optionally containing a passive artificially implanted or natural imaging reflectors (one of 150, 210, 310, 410, 630, 720) including a first, artificially implanted or natural stationary imaging reflective carrier element having a stationary ultrasound reflective surface (one of 140, 230, 330, 430, 640), and at least one second element, moveable relatively to the first element, artificially implanted or natural imaging reflective surface (one of 130, 210, 310, 410, 630, 720) with optionally at least at one end attached to said carrier element and oscillatable at a distance from said carrier element, said moveable surface is configured to be deflected by a pressure of a surrounding medium at an implantation site inside said body, wherein said surrounding medium is transparent for the imaging waves and a pressure and/or pressure changes of said medium are measureable at an implantation area of said reflector when implanted, and optionally, wherein said surrounding medium parts are not transparent for imaging waves, they can substitute optionally a first, stationary reflective carrier element and optionally the second, moveable reflective surface.

2. A system of claim 1 for non-invasive measurement of a pressure inside a body,
said system having an imaging unit, preferably ultrasound unit configured to measure intra body pressure, having at least one transducer, preferably ultrasound transducer (530, 650), arranged outside said body and radiating the beams (520, 660) into a target area inside said body, said system being configured to register the image time series estimating the volumes of the oscillating traceable regions in real time,

3. The system of claim 1 or 2, wherein said system optionally comprises a device of claim 1 when implanted at said target area, said device optionally having stationary and optionally moveable surface elements being the passive beam reflectors by the imaging unit, helping it to identify the oscillating traceable regions and configured to reflect said beams to said transducer, wherein said target site preferably is a cardiovascular target site and said pressure preferably is blood pressure, such as in blood vessels and/or said heart including a left atrium, right atrium, left ventricle, right ventricle,
said system further comprises a control unit configured to provide subsequent synchronous recordings of the pressure from alternative pressure meters, such as catheter based blood pressure sensors, such as with said imaging unit.

4. The system of claims 1 and 2 comprising a plurality of passive beam reflectors of claim 1, natural, or being implanted into a cardiovascular target area, such as blood vessels or areas of said heart, said reflectors having surface elements both optionally stationary and optionally moving under blood pressure changes and adapted to receive and reflect said beams.

5. The system of claim 2 or 3 further comprising an apparatus operatively connected to said transducer and adapted to send the beams to said one or more natural, or implanted beam reflectors and adapted to receive the beams reflected from said natural, or implanted one or more reflectors in return, configured to calculate said pressure measurements and comprising:
h) at least one probe, preferably ultrasound probe (530, 650) comprising at least one of said transducers for providing conversion of an electro-magnetic signal controlled by a control unit of said ultrasound apparatus into mechanical ultrasound signal to said ultrasound surfaces and said reverse conversion of said reflected ultrasound beam into electro-magnetic echoed signals provideable to said control unit for determination of intra body pressure, said echoed signals form the image time series used to calculate said pressure measurements;
i) at least one transmitter unit operatively connected to said control unit and configured to generate said electro-magnetic signals for further transformation into ultrasound beams by said transducer;
j) at least one receiver unit operatively connected to said control unit for said echoed signals;
k) at least one unit (540) for signal information processing being operatively connected to said control unit;
l) at least one unit for information data storage (540, 560) operatively connected to said control unit; and
m) at least one of said control unit, being a signal information processing unit adapted to run a control and calculation software and operatively connected to at least said beam former unit, transmitter unit, receive unit, alternative pressure meter unit during the calibrations and information storage unit (560).

6. The system of any of claims 2 to 4, further having said control unit configured to provide said subsequent synchronous recordings of the pressure measurements from alternative pressure meters, such as catheter based blood pressure sensors and imaging, preferably ultrasound measurements with the said probes and the calculation method for the best fit of the measured pressure values Pᵢ at implantation areas of said reflector and at time moments tᵢ as a function Pᵢ≈F(L₁ᵢ, L₂ᵢ) where L₁ᵢ is the brightness line of the first artificial or natural stationary surface (one of 140, 230, 330, 430, 640) in the image, which is preferably ultrasound image of said passive reflector and L₂ᵢ is the brightness line of the said second moving artificial or natural surface (one of 130, 210, 310, 410, 630, 720) in the image, which is preferably ultrasound image of said passive reflector respectively, measured at said time moment tᵢ.

7. The system of claim 6, wherein said control unit is configured to calculate said intra body pressure at said implantation area of said reflector as the best fitted through calibration function F, such that Pᵢ≈F (L₁ᵢ, L₂ᵢ) based on calibration measurements of said dependency Pᵢ from the parameters L₁ᵢ and L₂ᵢ.

8. The system of any of claims 1 to 7, wherein said reflector has a plate shape (630) and/or is a contoured membrane, such as a bent membrane having said apex, or a domed membrane, or a convex outer surface membrane (410), or a ball tipped narrow reflector (920) deformable under said intra body pressure changes when said reflector is implanted.

9. The system of any of claims 1 to 7, wherein said at least one passive beam reflector is deployable and implantable as a stand-alone device (150) without a medical implant carrier, such as implantable inside said pulmonary artery.

10. The system of any of claims 1-9, wherein said at least one passive beam reflector being attached or integral with a medical implantable device (720), (920), such as positioned at a distal and/or proximal end of an Atrial Flow Regulator (AFR) (230) device deployable to create a shunt between left and right atria of said heart, or such as positioned at a distal and/or proximal end of an Aorto-Pulmonary Flow Regulator (APFR) device deployable to create a shunt between a left pulmonary artery and a descending aorta.

11. The system of any of claims 1-10, wherein said at least one passive beam reflector (720), (920) being attached or integral with a medical implantable device, such as positioned at a distal and/or proximal end of a stent (910) deployed inside a vessel such as an artery such as Pulmonary Artery (PA) or deployed inside an interior channel of said AFR or APFR device (250) preferably implanted in said channel by using a same guide-wire for delivery.

12. The system of any of claims 1-7, wherein no passive beam reflectors are added to a medical implantable device deployed inside a vessel such as an artery such as Pulmonary Artery (PA) or inter-atrial septum, where the second natural moving surface is the opposite wall of the heart atrial chamber or Pulmonary Artery (PA).

13. The system of any of claims 1-7, wherein no medical implantable device is deployed inside a vessel such as an artery such as Pulmonary Artery (PA) or inter-atrial septum, where the first fixed natural surface and the second natural moving surface relatively to the fixed surface are existing anatomical structures such as opposite walls of the heart atrial chamber or Pulmonary Artery (PA).

14. The system of claims 3-13, wherein said control unit (540) is configured to perform said pressure measurements inside said body, such as blood pressure inside the cardiovascular system, by means of said at least one optional implantable passive ultrasound beam reflector when implanted and said ultrasound apparatus configured to operate in a time motion mode (TM- or M-mode) or the generalized M-mode being the set of M-modes corresponding to all ultrasound simultaneous beams, to register the brightnesses according to claims 5 and 6, of said first fixed artificial or natural and second artificial or natural moving surfaces of said one or more reflectors to said radiative surface of said ultrasound transducer denoted as L₁ and L₂, where L₁ is said brightness line of said first fixed natural or artificial surface of said artificial or natural passive reflector and L₂ is said brightness line of said second moving natural or artificial surface of said passive reflector respectively.

15. A method preferably based on the system of claims 1 to 14, for providing a pressure value P of a location inside a body, including
determining image brightnesses L₁, L₂ of the first fixed and second moving natural or artificial surfaces in a series of images of at least one optional passive ultrasound reflector previously implanted at a implantation area at said location inside said body and at a calculation time t by image processing, further including calculation of a local intra-body pressure P at time moments t as a function P=F(L₁, L₂) of a L₁, L₂ based on the calibration fit process, where the function P is being built by the subsequent synchronous recordings of the pressure measurements from alternative pressure meters, such as catheter based blood pressure sensors measurements and imaging measurements with the said transducers at the time moments tᵢ of said dependency of Pᵢ from L₁ᵢ, L₂ᵢ in a predetermined range and for prescribed shape of the function F.

16. A method preferably based on the system of claims 1 to 14, for providing a pressure value P of a location inside a body, including determining the brightnesses L₁, L₂, . . . , Lₙ of moving or fixed natural or artificial surfaces containing at least one optional passive reflector previously implanted at a implantation area at said location inside said body and at a measurement time t by image processing, and further including calculation of a local intra-body pressure P at time moments t as a function Pᵢ≈F(L₁, L₂, . . . , Lₙ) of L₁, L₂, . . . , Lₙ based on the calibration fit process, where the function P is preferably being built by the measurements as in claim 5 at time moments tᵢ of said dependency of Pᵢ from L₁ᵢ, L₂ᵢ, . . . , Lₙᵢ in a predetermined range and for prescribed shape of the function F.

17. The method of claim 15 providing a pressure value P of a location inside a body, including determining the brightnesses L₁, L₂, . . . , Lₙ of moving or fixed natural or artificial surfaces containing at least one optional passive ultrasound reflector previously implanted at a implantation area at said location inside said body and at a measurement time t by image processing, further including calculation of a local pressure P at time moments t as a function P≈F(L₁, L₂, . . . , Lₙ) of L₁, L₂, . . . , Lₙ based on the calibration fit process, where the function P is the linear function of the brightnesses L₁, L₂, . . . , Lₙ with the coefficients W₁, W₂, . . . , Wₙ optimally fitted for equations Pᵢ≈W₁L₁ᵢ+ . . . +WₙLₙᵢ C to hold for the subsequent recordings from claim 5. Said coefficients W₁, W₂, . . . , Wₙ are proportional to the cut areas of the said target volume at given depths orthogonally to the transducer working plane, while the whole sum is approximating the pressure as the function of the target area volume.

18. The method of claim 15 providing a pressure value P of a location inside a body, including determination the brightnesses L₁, L₂, . . . , Lₙ, from claim 16 which at the measurement time moments tᵢ are equal to L₁ᵢ, L₂ᵢ, Lₙᵢ and are representing either the brightness peak path across the joint horizontally averaged ultrasound images Iᵢ, or the most correlated normalized brightness lines to {L₁ᵢ} and respectively {Lₙᵢ} in the set {Iᵢ}, where {L₁ᵢ} and {Lₙᵢ} are the upper and lower brightness peak paths across {Iᵢ}.

19. The method of claim 14 or 16 including measuring said pressure inside the cardiovascular system, said pressure being intravascular blood pressure, such as inside a blood vessel or the heart, by means of at least one implantable passive ultrasound beam reflector (630), which reflects the Ultrasound waves emitted by Ultrasound apparatus (530, 650) configured to work in the time motion mode (TM- or M-mode) or the generalized M-mode being the set of M-modes corresponding to all ultrasound simultaneous beams, in order to register the brightnesses, according to claims 5 and 6 and including a 2-Dimensional (2D- or B-) visualization mode, used for visualization of said target area of said cardiovascular system having said passive artificially implanted or natural ultrasound beam reflector, based on a change of said distances in dependency of said changes of said pressure at said target area.

20. The method of claim 12, further including determining the blood flow velocities in an operational mode of said ultrasound apparatus including a spectral Doppler mode (D-mode) with said visualization of said part of said cardiovascular system including said at least one passive artificially implanted or natural ultrasound beam reflector in the 2-Dimensional (2D- or B-) mode and with measurements of velocities of blood flow in said D-mode.

21. The method of claims 14 to 16, including
a. setting said transducer (530, 650) into operation; providing a user interface (540), such as a graphical user interface (GUI) including an on-screen image, and displaying, and setting a first operation mode run in B-mode, forming an ultrasound picture in said user interface;
b. pointing said transducer in a direction to said target implantation area where said artificially implanted or natural reflector for pressure measurement is located inside said body, and holding said position and/or adjusting said direction according to said displayed image until said reflector (630) is visible on said image;
c. switching said ultrasound apparatus (530, 650) to a second mode of operation, including but not limited to an M-mode, or the generalized M-mode being the set of M-modes corresponding to all ultrasound simultaneous beams, and retrieving pressure based reflected or echoed signal changes from said reflector for a certain time length, and calculated said pressure inside said body based on said retrieved reflected signal changes.

22. The method of any of claims 14-16, including adjusting said measurements to 3-dimensional movements of said passive artificially implanted or natural ultrasound beam reflector (210), and/or a medical implant to which said reflector is associated, such as an AFR or APFR device (230).

23. Ultrasound probe of an ultrasound apparatus (530, 650) included in any of claims 2-15, including a single-element wide-band multi-frequency transducer configured to perform of said measurements of said blood pressure in said blood vessels or the heart chambers in accordance with any of claims 9-16.

24. Ultrasound probe of claim 23 having two acoustically and electrically separated wide-band multi-frequency transducers, one of which works as a radiator of said ultrasound signals and said second works as said receiver of said echoed-signals to said ultrasound apparatus performing said measurements of said pressure in accordance with any of claims 15-22, wherein said multi-element wide-band multi-frequency transducers preferably are piezo-electric transducers.

25. A system for performing said method of claim 15-22, including:
i) an ultrasound apparatus with a communication interface (530, 650),
ii) a client computer or handheld device with a client software application installed (540),
iii) optional local medical centre server (560), and
iv) optional cloud information storage (560).

26. A software including code segments for
d. setting an ultrasound transducer (530, 650) into operation; providing on a user interface, such as a graphical user interface (GUI) including an on-screen image, and displaying, and setting a first operation mode run in B-mode, forming an ultrasound picture in said user interface (540);
e. displaying image until said reflector is visible (510, 630) on said image when said transducer is pointed in a direction to said target implantation area where said reflector for pressure measurement is located inside said body;
f. switching said ultrasound apparatus (530, 650) to a second mode of operation, including an M-mode, or the generalized M-mode being the set of M-modes corresponding to all ultrasound simultaneous beams, and retrieving pressure based reflected or echoed signal changes from said reflectors (510, 630) for a certain time length, and calculated said pressure inside said body based on said retrieved reflected signal changes.

27. A medical procedure for deployment said passive ultrasound beam reflector of claim 1 inside the cardiovascular system, said procedure comprising
(a) deployment (810) of said passive ultrasound beam reflector inside a sheath being attached by means of a proximal end (220, 320, 420) to a capturing unit, being arranged at a distal end of a delivery unit for releasable attaching of said passive ultrasound beam reflector to said capturing unit;
(b) endovascular transportation (820) of said carrier unit to an appropriate heart region inside said sheath by means of guide wire manipulations;
(c) orientation (830) of said carrier unit by means of said guide wire manipulations inside the cardiovascular system according to fiducial marks on said capturing unit and said delivery unit, such as visible on ultra-sound or fluoroscopy equipment
(d) anchoring (840) of said passive ultrasound beam reflector;
(e) releasing (850) said carrier unit from said capturing unit of said delivery unit; and
(f) extracting (860) of said sheath from the heart and said body.

28. A system, such as of claim 1, for determining a pressure inside a body, including a control unit configured to
estimate at least a volume of an oscillating traceable region inside said body from at least a series of images generated by an ultrasound or other medical imaging unit, and configured to correlate said volume with a pressure at said region for said determining of said pressure.

29. The system of claim 28, including at least one medical implant previously implanted at said region for tracing said oscillating region in said series of images; said implant optionally having at least one imaging reflective surface.

30. The system of claim 29, said medical implant being implantable in an atrial cardiac region, such as including at least one of an ASD occluder, PFO occluder, LAA occluder, Atrial shunt device, Paravalvular leakage occluder; and said pressure is a pressure in at least one atrium of said heart.

31. A method, such as of claims 15-22, for determining a pressure inside a body, including estimating at least a volume of an oscillating traceable region inside said body from at least a series of images generated by an ultrasound or other medical imaging unit, and correlating said volume with a pressure at said region for said determining of said pressure.

32. A software, such as of claims 25-26, for performing the method of claim 31, preferably stored on a computer readable medium.

33. A medical implant, such as comprised in claim 1, being implantable in an atrial cardiac region, such as including at least one of an ASD occluder, PFO occluder, LAA occluder, Atrial shunt device, Paravalvular leakage occluder; having at least one imaging reflective surface attached thereto for determining a pressure in at least one atrium of said heart.

34. Use of a previously implanted medical implant, such as of claim 33, in a system of claims 1-14.
